(19) United States
(12) Patent Application Publication
(10) Pub. No.: US 2026/0272640 A1
(43) Pub. Date: Sep. 17, 2026

(54) PROSTHETIC VALVES AND VALVULAR STRUCTURES THEREOF

(71) Applicant: Edwards Lifesciences Corporation, Irvine, CA (US)

(72) Inventor: Tamir S. Levi, Zikhron Yaakov (IL)

(73) Assignee: Edwards Lifesciences Corporation, Irvine, CA (US)

(21) Appl. No.: 19/667,532

(22) Filed: May 4, 2026

Related U.S. Application Data
(63) Continuation of application No. PCT/US2024/055623, filed on Nov. 13, 2024.
(60) Provisional application No. 63/548,424, filed on Nov. 14, 2023.

(51) Int. Cl. A61F 2/24

(52) U.S. Cl. CPC A61F 2/2415; A61F 2/2418; A61F 2230/0069

(57) ABSTRACT

The present disclosure relates to prosthetic valves and valvular structures thereof. In an example, a prosthetic valve can include a frame movable between a radially compressed state and a radially expanded state, coupled to the frame and comprising a plurality of valvular segments defining leaflets configured to regulate flow through the prosthetic valve. Each valvular segment can include a leaflet and an attachment strip segment. The leaflet can have a curved cusp line, a free edge proximal to the cusp line, and a leaflet belly extending between the cusp line and the free edge, and the attachment strip segment can extend from the cusp line to a curved terminal edge. The attachment strip and the leaflet of each valvular segment are integrally formed from a one-piece material, wherein the leaflet belly is not flattenable while the attachment strip segment is flattenable.

### CROSS-REFERENCE TO RELATED APPLICATIONS

This application is a continuation of International Application No. PCT/US2024/055623, filed Nov. 13, 2024, which claims the benefit of U.S. Provisional Application No. 63/548,424, filed Nov. 14, 2023, which is incorporated by reference herein.

### FIELD

The present disclosure relates to prosthetic valves, and in particular, to prosthetic valves having valvular structures that can include non-flattenable movable leaflet bellies.

### BACKGROUND

Native heart valves, such as the aortic, pulmonary and mitral valves, function to assure adequate directional flow from and to the heart, and between the heart's chambers, to supply blood to the whole cardiovascular system. Various valvular diseases can render the valves ineffective and require replacement with artificial valves. Surgical procedures can be performed to repair or replace a heart valve. Surgeries are prone to an abundance of clinical complications, hence alternative less invasive techniques of delivering a prosthetic heart valve over a catheter and implanting it over the native malfunctioning valve, have been developed over the years.

Different types of prosthetic heart valves are known to date, including balloon expandable valve, self-expandable valves and mechanically-expandable valves. Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, towards the native malfunctioning valve. Once the prosthetic valve is properly positioned at the desired site of implantation, it can be expanded against the surrounding anatomy, such as an annulus of a native valve, and the delivery assembly can be retrieved thereafter.

### SUMMARY

Described herein are prosthetic valves and method of manufacturing of at least some components of the prosthetic valves, such as valvular structures configured to be mounted inside frames of the prosthetic valves, and method assembling and/or attaching the valvular structures to the frames.

According to some aspects of the disclosure, there is provided a prosthetic valve comprising a frame movable between a radially compressed state and a radially expanded state, and a valvular structure coupled to the frame. The valvular structure comprises a plurality of valvular segments defining leaflets configured to regulate flow through the prosthetic valve. Each valvular segment comprises a leaflet and an attachment strip. The leaflet has a curved cusp line, a free edge proximal to the cusp line, and a leaflet belly extending between the cusp line and the free edge. The attachment strip segment extends from the cusp line to a curved terminal edge of the attachment strip segment.

In some examples, the attachment strip and the leaflet of each valvular segment are integrally formed from a one-piece material.

In some examples, the leaflet belly is not flattenable, while the attachment strip segment is flattenable.

In some examples, the attachment strip segment defines a width in a range of 0.5 to 3 mm.

In some examples, the width of the attachment strip segment is in a range of 1 to 2 mm.

In some examples, the valvular structure is a one-piece material, wherein the plurality of attachment strip segments together form a continuous one-piece attachment strip of the valvular structure.

In some examples, the attachment strip segments are coupled to the frame.

In some examples, each attachment strip segment is placed against an inner surface of the frame, such that the terminal edge is closer to an inflow end of the frame than the cusp line.

In some examples, each attachment strip segment is folded upwardly towards an outflow end of the frame and against an inner surface of the frame, such that the terminal edge is closer to the outflow end of the frame than the cusp line.

In some examples, the valvular structure further comprises commissures formed by commissure attachment regions of the valvular structure coupled to the commissure posts of the frame.

In some examples, the free edges are distally offset by an offsetting height defined between proximal ends of the commissure attachment regions and a distal-most level of the free edges.

In some examples, the offsetting height is equal to or greater than 2 mm.

In some examples, the offsetting height is not greater than 5 mm.

According to some aspects of the disclosure, there is provided a method comprising placing a tissue patch between a first template and a second template of a mold assembly, and forming the tissue patch to include at least one leaflet belly which is not flattenable and at least one attachment strip segment with is flattenable.

In some examples, forming the leaflet belly comprises pressing a first part the tissue patch between a raised leaflet forming portion of the first template and a recessed leaflet forming portion of the second template.

In some examples, forming the leaflet belly further comprises applying a cross-linking solution to the tissue patch.

In some examples, forming the attachment strip segment comprises trimming a second part of the tissue patch that was pressed between a planar segment of the first template and a planar segment of a second template, along a curved terminal edge, such that the resulting attachment strip segment extends between the curved terminal edge and a cusp line that separates between the attachment strip segment and the leaflet belly.

In some examples, the trimming defines a width of the attachment strip segment in a range of 0.5 to 3 mm.

In some examples, the trimming defines a width of the attachment strip segment in a range of 1 to 2 mm.

In some examples, the first template comprises a plurality of raised leaflet forming portions and the second template comprises a plurality of recessed leaflet forming portions.

In some examples, the tissue patch is a one-piece tissue patch, and wherein the at least one leaflet belly comprises a plurality of leaflet bellies of the one-piece tissue patch.

In some examples, the at least one attachment strip segment comprises a plurality of attachment strip segments together defining a continuous attachment strip of the one-piece tissue patch.

In some examples, the method further comprises forming a valvular structure from the one-piece tissue patch by coupling side edges of the tissue patch to each other.

In some examples, the method further comprises coupling the attachment strip segments to a frame of a prosthetic valve.

In some examples, the coupling comprises placing each attachment strip segment, against an inner surface of the frame, such that the terminal edge is closer to an inflow end of the frame than the cusp line.

In some examples, the coupling comprises folding each attachment strip segment upwardly towards an outflow end of the frame and against an inner surface of the frame, such that the terminal edge is closer to the outflow end of the frame than the cusp line.

In some examples, the coupling comprises passing a suture that connect the attachment strip segment to the frame, through a reinforcement member placed against a side of the attachment strip segment opposite to the frame.

According to some aspects of the disclosure, there is provided a prosthetic valve comprising a frame movable between a radially compressed state and a radially expanded state, and a valvular structure coupled to the frame. The frame comprises an outflow rung of angled struts extending between outflow apices and lower junctions of the outflow rung, and a plurality of commissure posts proximally extending from the lower junctions of the outflow rung. The valvular structure comprises a plurality of leaflets configured to regulate flow through the prosthetic valve. Each leaflet comprises a curved cusp line, a free edge proximal to the cusp line, and a leaflet belly extending between the cusp line and the free edge. The valvular structure further comprises a plurality of commissure attachment regions between adjacent leaflet bellies, each commissure attachment region terminating at a proximal end. The commissure attachment regions are coupled to the commissure posts of the frame to define commissures of the valvular structure. The free edges of the leaflets are distally offset by an offsetting height defined between the proximal ends of the commissure attachment regions and a distal-most level of the free edges.

In some examples, the leaflet belly is not flattenable.

In some examples, the offsetting height is equal to or greater than 2 mm.

In some examples, the offsetting height is not greater than 5 mm.

In some examples, the valvular structure is a one-piece material.

According to some aspects of the disclosure, there is provided a method comprising placing a tissue patch between a first template and a second template of a mold assembly, and forming at least one leaflet belly between commissure attachment regions of the tissue patch by pressing part the tissue patch between a raised leaflet forming portion of the first template and a recessed leaflet forming portion of the second template.

In some examples, the forming further comprises applying a cross-linking solution to the tissue patch, such that the resulting leaflet belly is not flattenable.

In some examples, the method further comprises trimming the leaflet patch between the commissure attachment regions on both sides of the leaflet belly, thus forming a free edge of the leaflet belly which is distally offset by an offsetting height defined between proximal ends of the commissure attachment regions and a distal-most level of the free edges.

In some examples, the method further comprises attaching the commissure attachment regions to commissure posts of a frame of a prosthetic valve, wherein the commissure posts extend proximally from lower junctions of an outflow rung of angled struts of the frame.

In some examples, the offsetting height is equal to or greater than 2 mm.

In some examples, the offsetting height is not greater than 5 mm.

In some examples, the tissue patch is rectangularly shaped prior to the placing.

In some examples, the first template comprises a plurality of raised leaflet forming portions and the second template comprises a plurality of recessed leaflet forming portions.

In some examples, the tissue patch is a one-piece tissue patch, and wherein the at least one leaflet belly comprises a plurality of leaflet bellies of the one-piece tissue patch.

In some examples, the method further comprising, after the trimming and before the attaching the commissure attachment regions, forming a valvular structure from the one-piece tissue patch by coupling side edges of the tissue patch to each other.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

### In the Figures

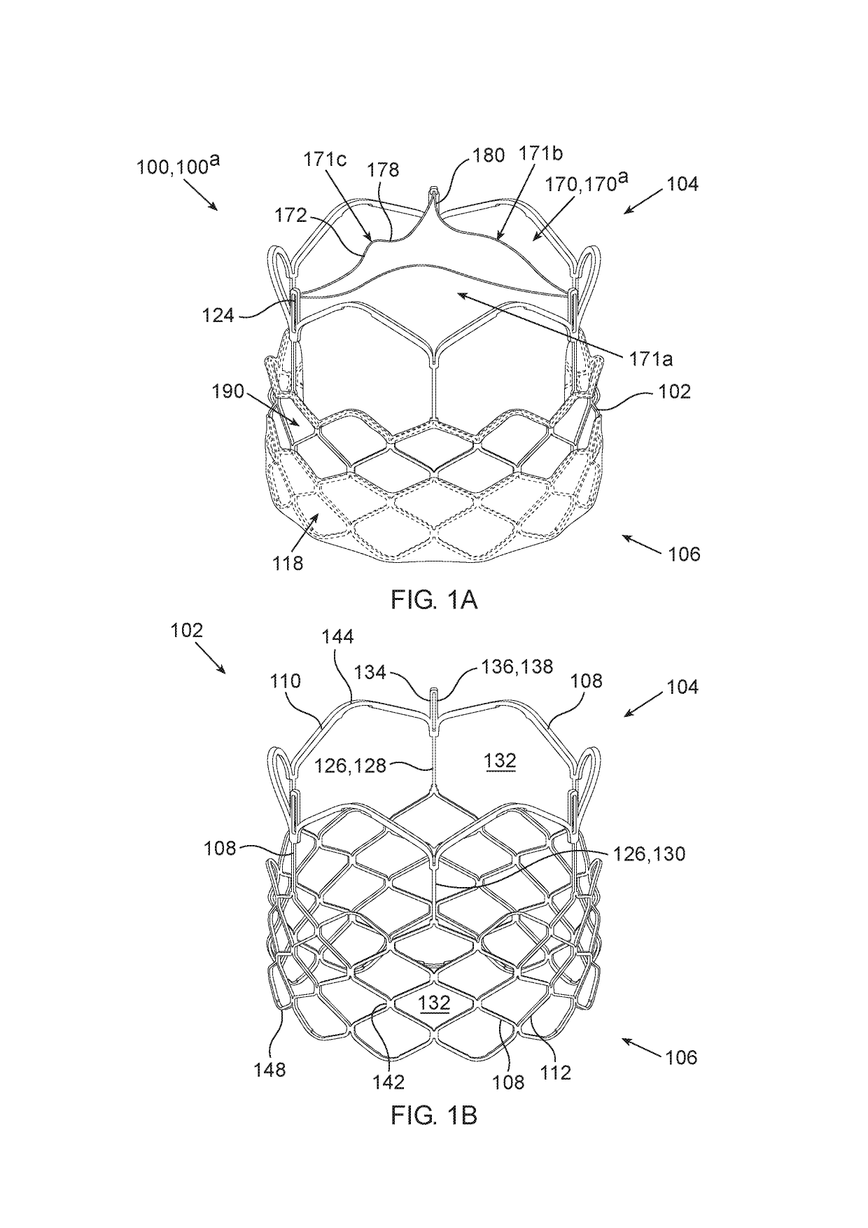

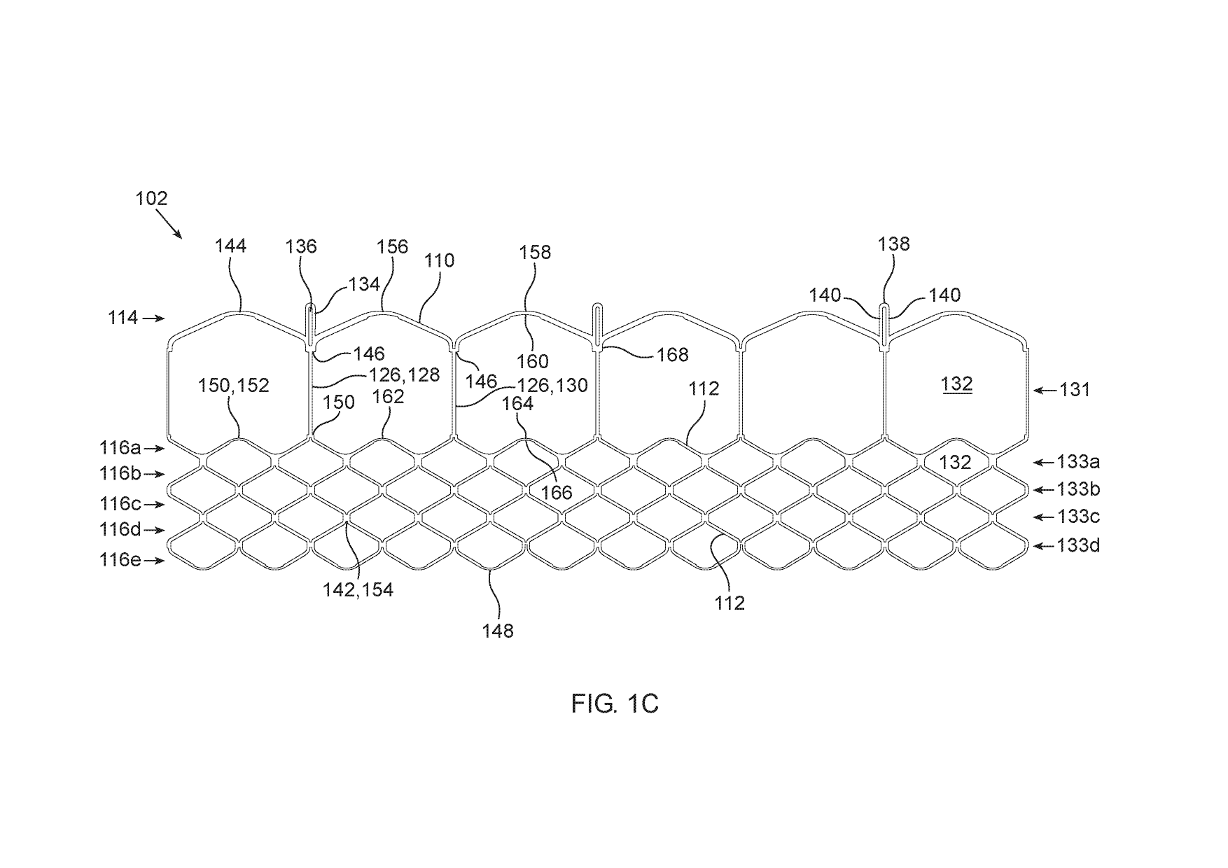

FIG. 1A is a perspective side view of an exemplary prosthetic valve.

FIG. 1B is a perspective view of an annular frame of the prosthetic valve of FIG. 1A.

FIG. 1C is a flattened view of the frame of FIG. 1B.

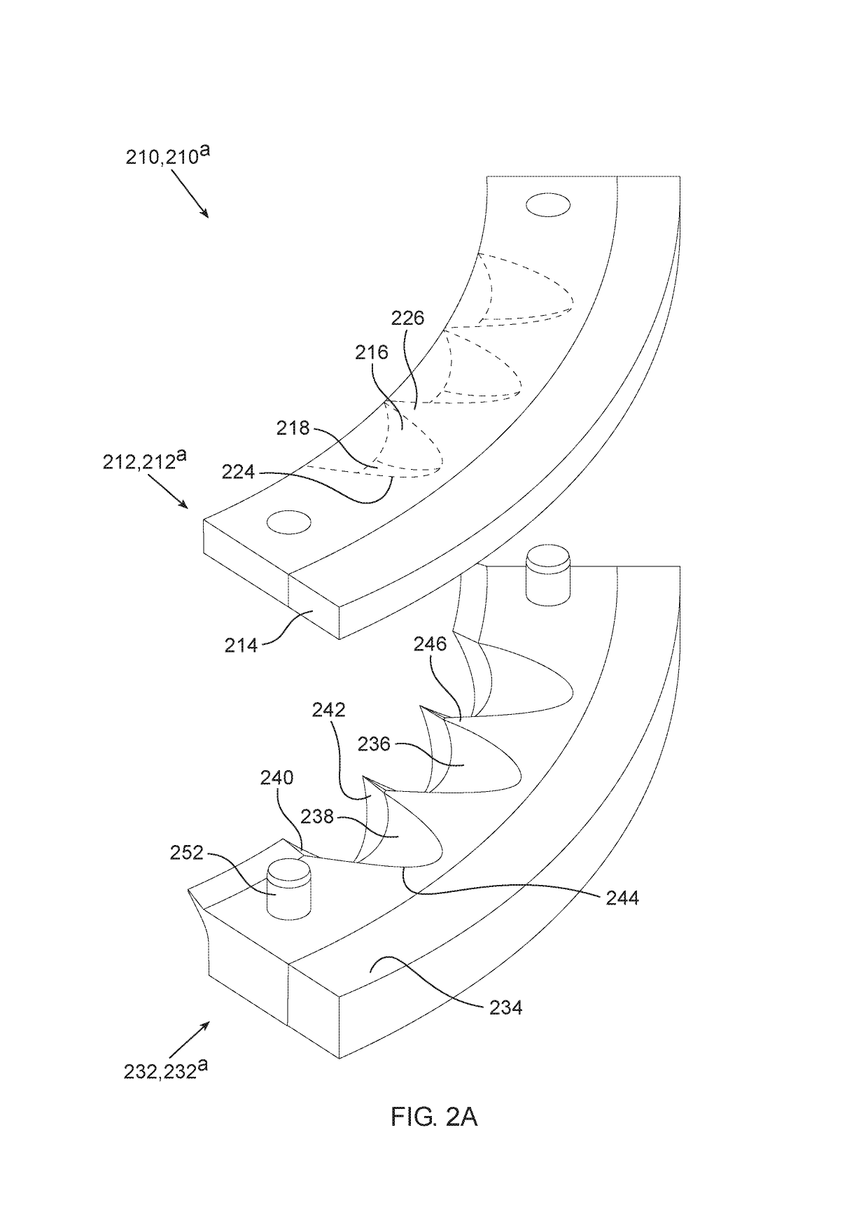

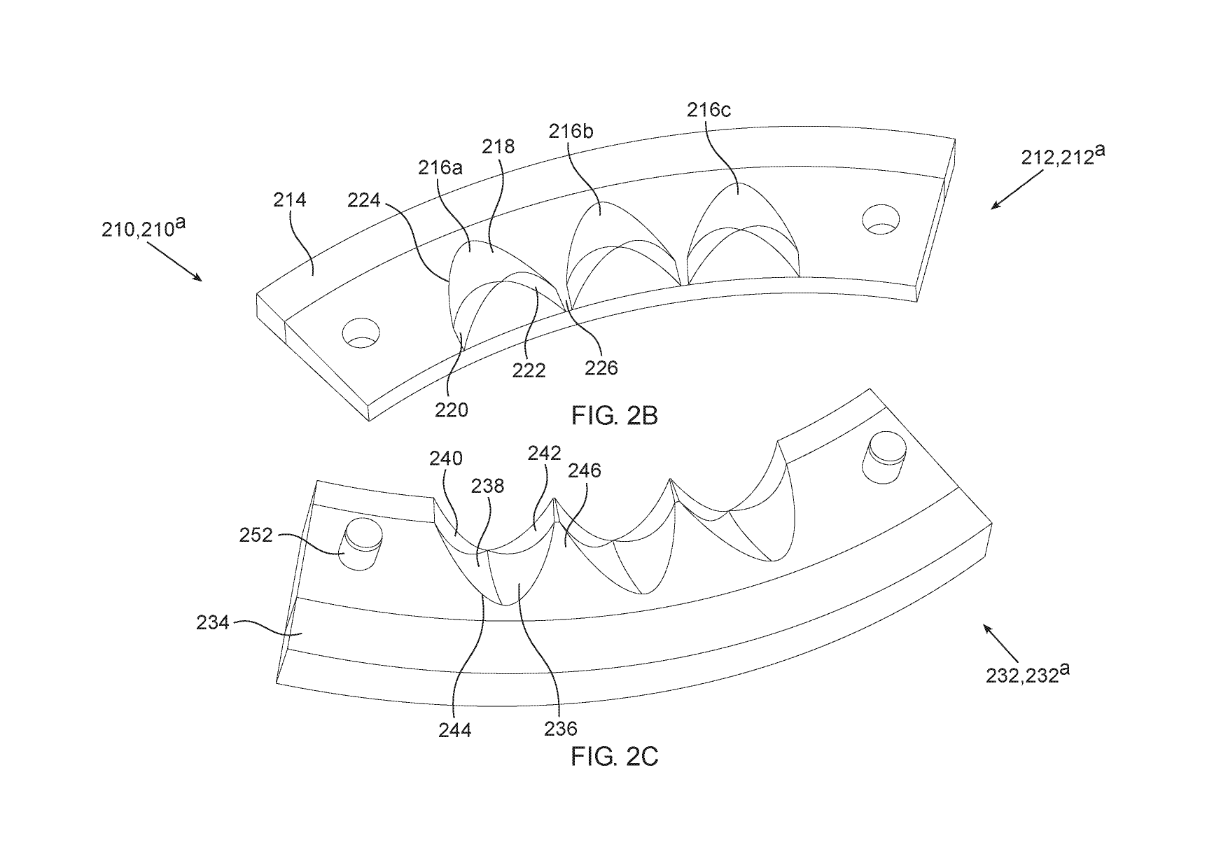

FIGS. 2A-2C show parts of an exemplary mold assembly that can be used in a method for fabricating a valvular structure.

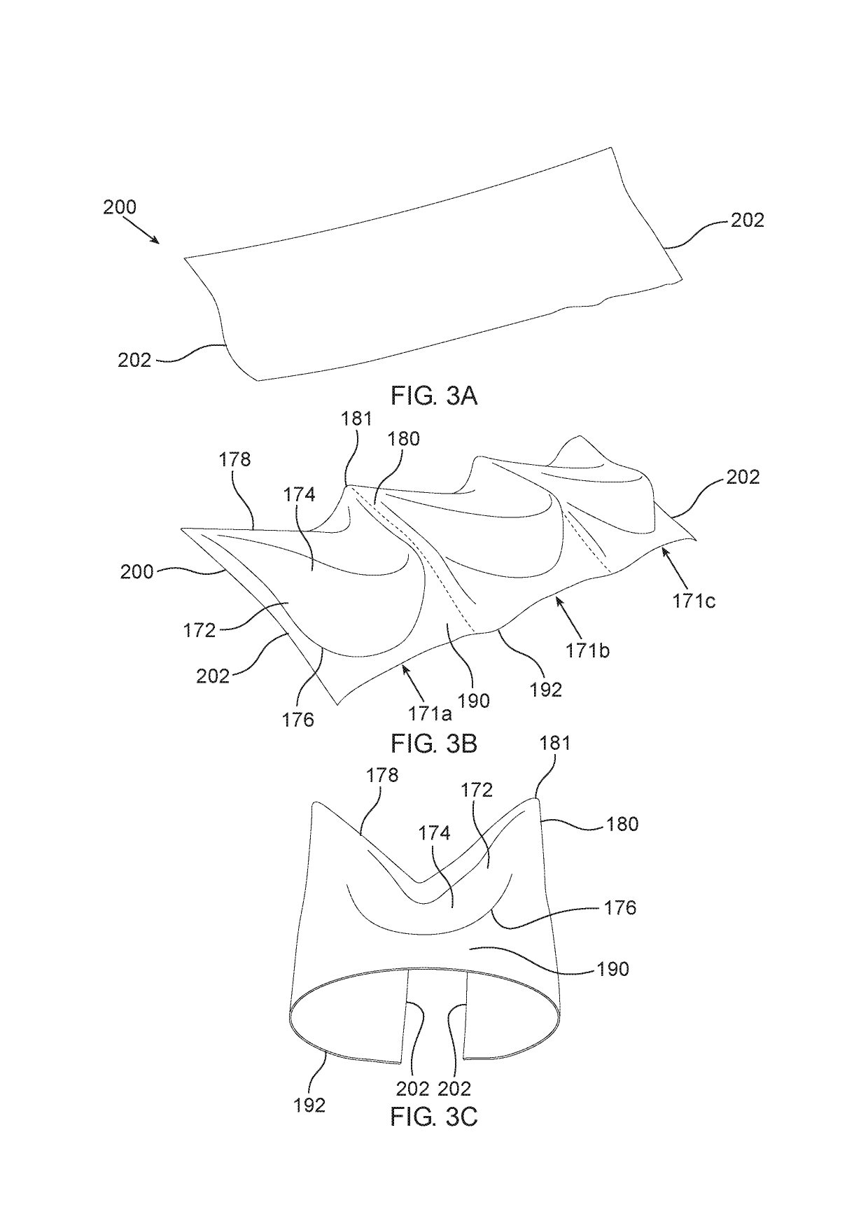

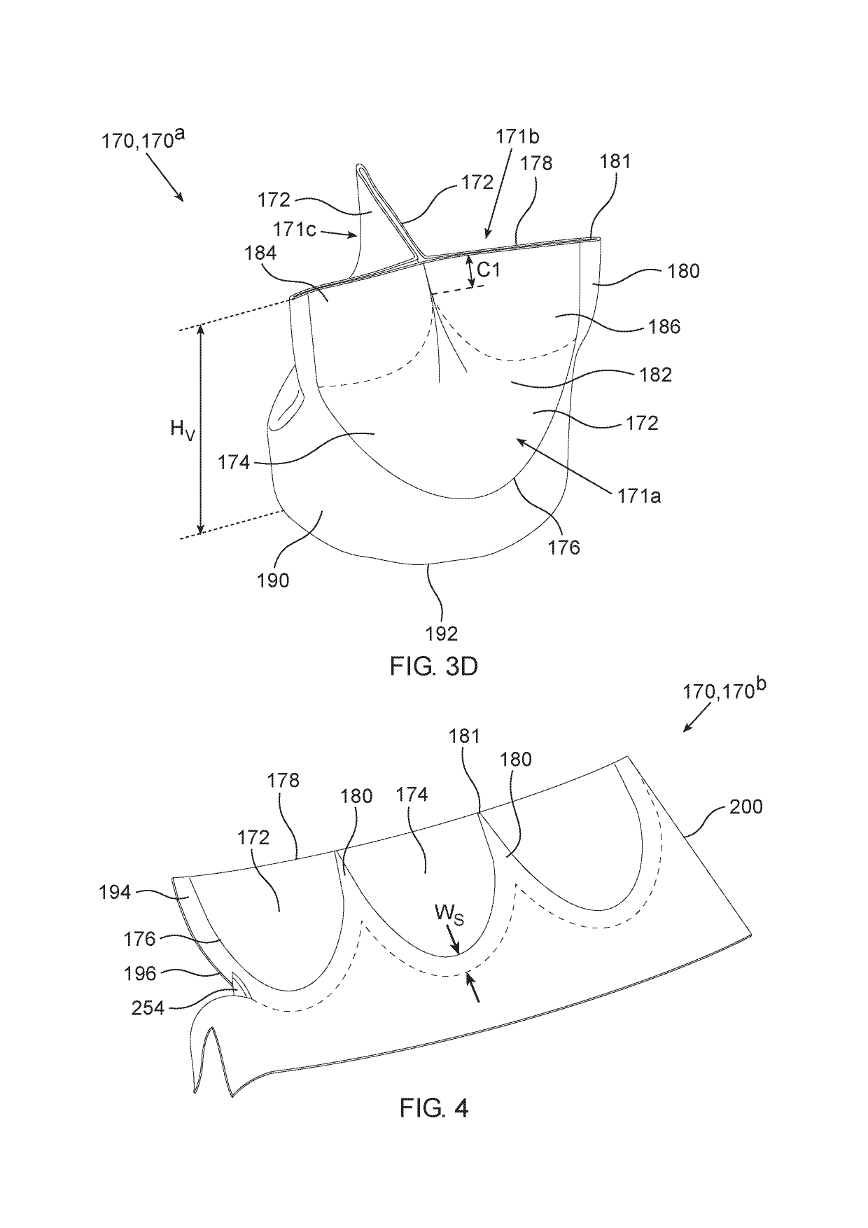

FIGS. 3A shows a patch of material prior to placement thereof in a mold assembly.

FIG. 3B shows the 3D-shaped patch removed from a mold assembly.

FIG. 3C shows an optional step in a method for forming a tubular valvular structure by rolling the 3D-shaped patch.

FIG. 3D shows an exemplary valvular structure that includes an engagement portion.

FIG. 4 shows a patch trimmed to form an attachment strip of the valvular structure.

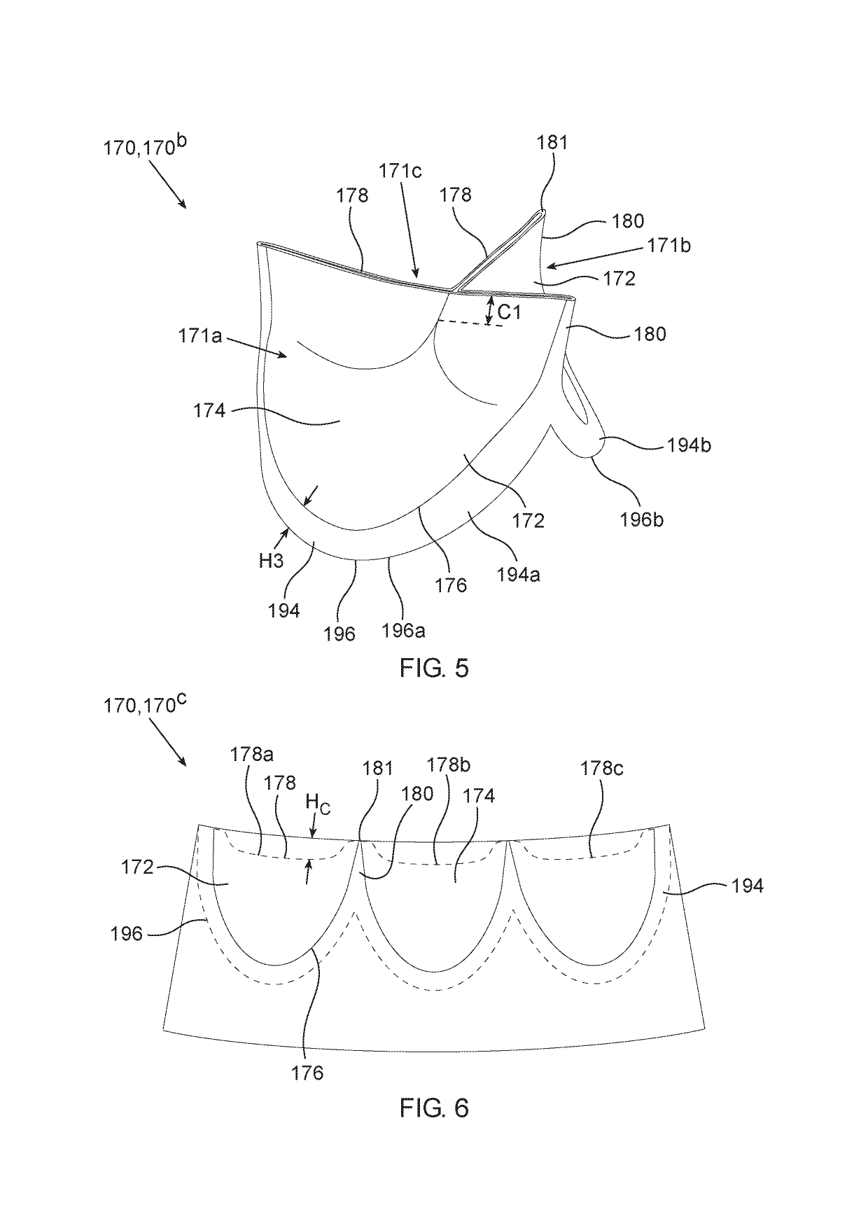

FIG. 5 shows an exemplary valvular structure that includes an attachment strip.

FIG. 6 shows a patch trimmed to form free edges which are distally offset from the proximal ends of commissure attachment regions.

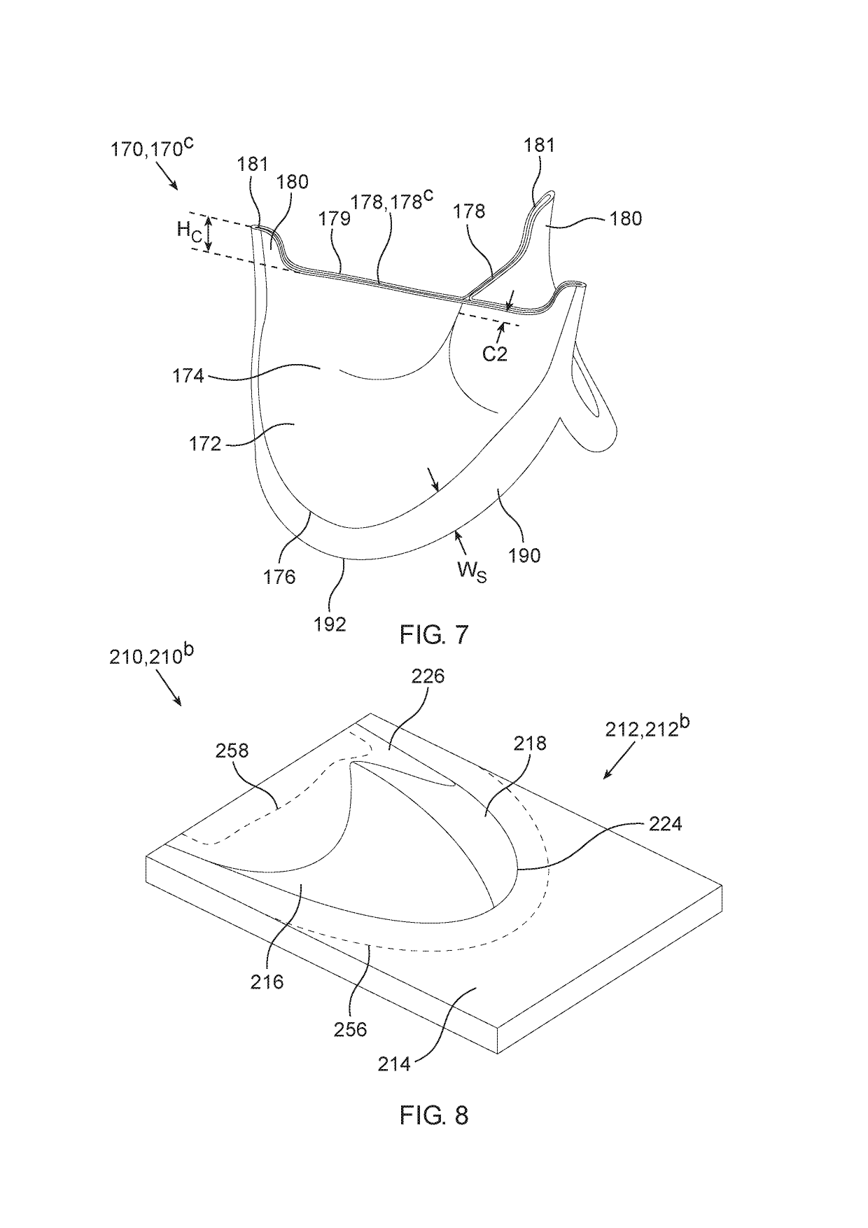

FIG. 7 shows an exemplary valvular structure that includes free edges which are distally offset from the proximal ends of commissure attachment regions.

FIG. 8 is a perspective view of a first template of an exemplary mold assembly that can be used to form a single valvular section.

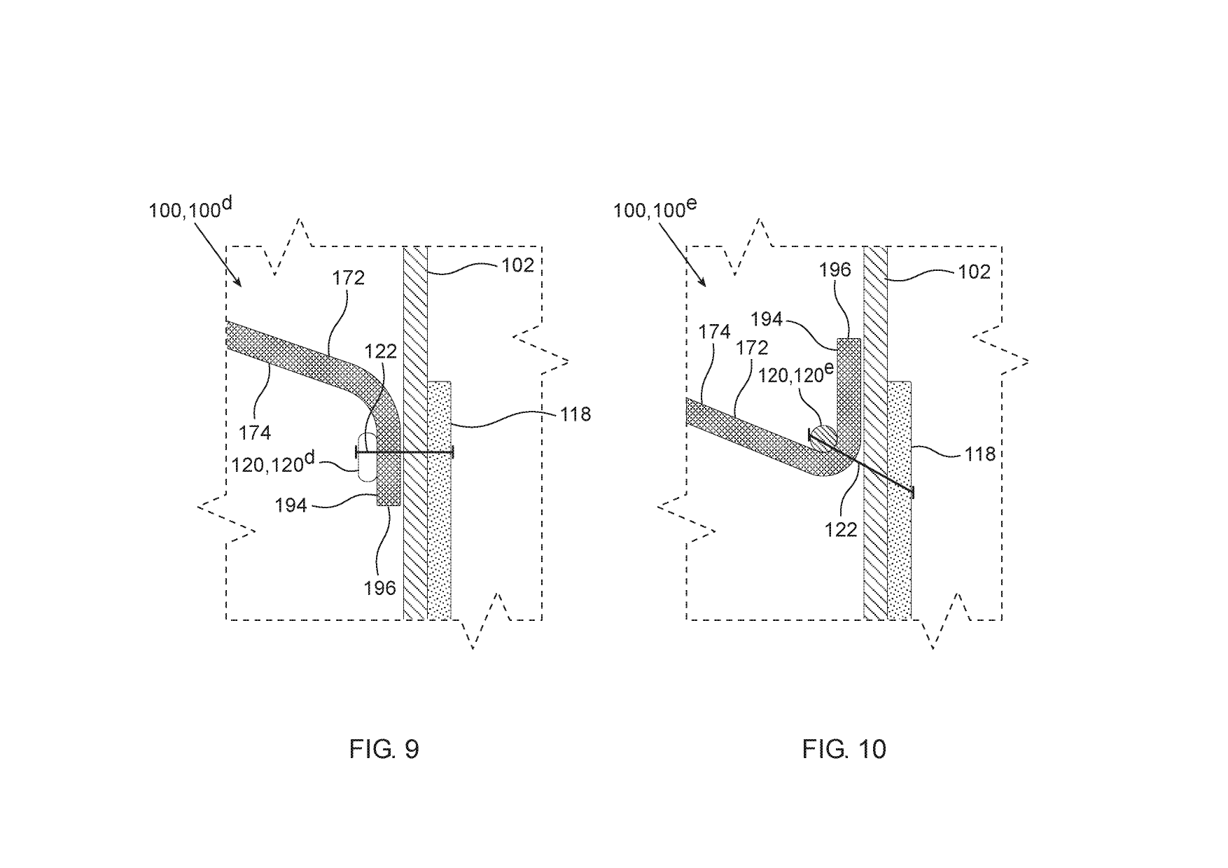

FIG. 9 shows a cross-sectional view of a portion of an exemplary prosthetic valve, in which an attachment strip extends downwardly towards the inflow end.

FIG. 10 shows a cross-sectional view of a portion of an exemplary prosthetic valve, in which an attachment strip is folded upwardly towards the outflow end.

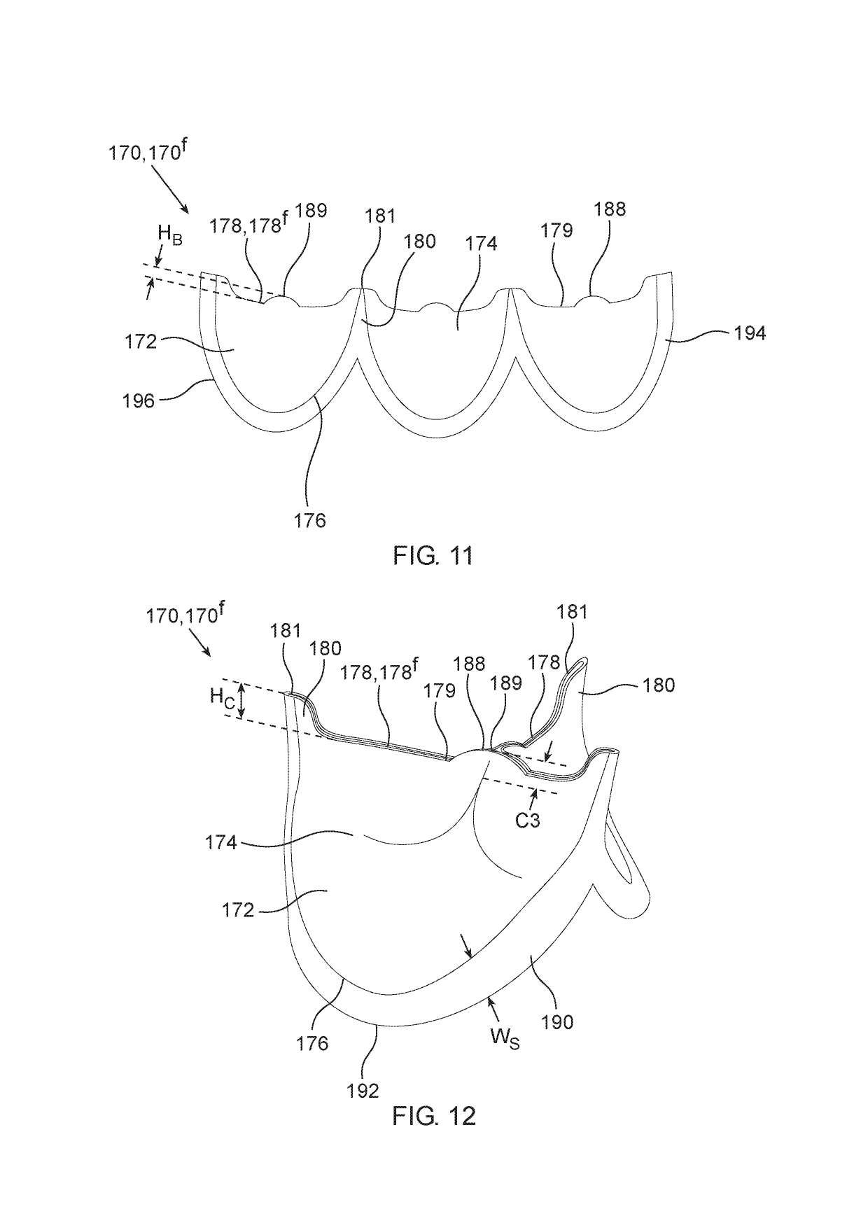

FIG. 11 shows a flattened configuration of an exemplary valvular structure that includes bumps or protrusions along the free edges.

FIG. 12 shows the valvular structure of FIG. 11 is an assembled configuration.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms "have" or “includes” means “comprises”. Further, the terms “coupled”, “connected”, and "attached", as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The term “proximal”, as used herein, generally refers to a position, direction, or portion of any device or a component of a device, which is closer to a user of a delivery apparatus that can be used to implant the device in the patient and farther away from the implantation site. The term “proximal” when used with two spatially separated positions or parts of an object can be understood to mean closer to or oriented towards the proximal end of the device. The term “distal”, as used herein, generally refers to a position, direction, or portion of any device or a component of a device, which is further away from the user and closer to the implantation site. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

FIGS. 1A and 1B show perspective views of an example of a prosthetic valve 100, with and without soft components (such as skirts and a leaflet assembly), respectively. The term "prosthetic valve", as used herein, refers to any type of a prosthetic valve deliverable to a patient's target site over a catheter, which is radially expandable and compressible between a radially compressed, or crimped, state, and a radially expanded state. Thus, the prosthetic valves can be crimped on or retained by an implant delivery apparatus (not shown) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve 100 of the current disclosure may include any prosthetic valve configured to be mounted within the native aortic valve, the native mitral valve, the native pulmonary valve, and the native tricuspid valve.

It is to be understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus. Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Patent No. 10,603,165, International Application No. PCT/US2021/052745, and U.S. Provisional Application Nos. 63/085,947 and 63/209904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

The term "plurality", as used herein, means more than one.

The prosthetic valve 100 comprises an inflow end 106 and an outflow end 104. In some instances, the inflow end 106 is the distal end of the prosthetic valve 100, and the outflow end 104 is the proximal end of the prosthetic valve 100. Alternatively, depending for example on the delivery approach of the valve, the inflow end can be the proximal end of the prosthetic valve, and the outflow end can be the distal end of the prosthetic valve.

The term "outflow", as used herein, refers to a region of the prosthetic valve through which the blood flows through and out of the prosthetic valve 100.

The term "inflow", as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

The terms “longitudinal” and “axial”, as used herein, refer to an axis extending in the proximal and distal directions, unless otherwise expressly defined.

The valve 100 comprises an annular frame 102 movable between a radially compressed state and a radially expanded state, and a valvular structure 170 mounted within the frame 102. FIG. 1C shows the frame 102 of FIG. 1B in a flat configuration for purposes of illustration. The frame 102 can be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When constructed of a plastically-deformable materials, the frame 102 can be crimped to a radially compressed state on a balloon catheter (not shown), and then expanded inside a patient by an inflatable balloon or equivalent expansion mechanism. Alternatively or additionally, the frame 102 can be made of shape-memory materials such as, but not limited to, nickel titanium alloy (e.g., Nitinol).

In the example illustrated in FIGS. 1A-1B, the frame 102 is an annular, stent-like structure comprising a plurality of intersecting struts 108. In this application, the term "strut" 108 encompasses vertical struts, angled or curved struts, support posts, commissure windows, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define a plurality of cells 132 arranged in several cell rows 131, 133. The frame 102 can have a cylindrical or substantially cylindrical shape having a constant diameter from the inflow end 106 to the outflow end 104 as shown, or the frame can vary in diameter along the height of the frame, as disclosed in US Pat. No. 9,155,619, which is incorporated herein by reference.

Two or more struts 108 can intersect at junctions 142, which can be can be equally or unequally spaced apart from each other. The struts 108 may be pivotable or bendable relative to each other, so as to permit frame expansion or compression. For example, the frame 102 can be formed from a single piece of material, such as a metal tube, via various processes such as, but not limited to, laser cutting, electroforming, and/or physical vapor deposition, while retaining the ability to collapse/expand radially in the absence of hinges and like.

FIGS. 1A-1C show an exemplary prosthetic valve 100 that can be representative of, but is not limited to, a balloon expandable prosthetic valve. In some examples, the struts 108 can include a plurality of angled struts and vertical or axial struts. The term "axial strut" refers to a strut or a component of the frame 102 that generally extends in an axial direction, while the term "angled strut" generally refers to a strut that can extend at an angle relative to an axial line intersecting therewith along a plane defined by the frame 102. It is to be understood that the term "angled strut" encompasses both linear angled struts and curved struts.

The frame 102 of exemplary prosthetic valve 100 illustrated in FIGS. 1A-1C comprises an outflow rung 114 of angled struts 110, and a plurality of subsequent rungs 116 of angled struts 112, wherein the subsequent rungs 116 are distal to the outflow rung 114. In such examples, the struts may be pivotable or bendable relative to each other, so as to permit frame expansion or compression. For example, the frame 102 can be formed from a single piece of material, such as a metal tube, via various processes such as, but not limited to, laser cutting, electroforming, and/or physical vapor deposition, while retaining the ability to collapse/expand radially in the absence of hinges and like. In the examples illustrated in FIGS. 1A-1C, the frame 102 is shown to include five rungs 116a, 116b, 116c, 116c, 116d, and 116e, of angled struts 112. It is to be understood that any frame disclosed herein can include any other plurality of subsequent rungs 116 of angled struts 112, such as two, three, four, or more than five subsequent rungs.

The frame 102 of exemplary prosthetic valve 100 illustrated in FIGS. 1A-1C is shown to further include a plurality of axial struts 126 vertically extending between the outflow rung 114 of angled struts 110, and the first or proximal-most subsequent rung 116a of angled struts 112.

The upper end portions of the angled struts 110 of the outflow rung 114 are forming apices 144 at or proximate to the outflow end 104 of the valve 100, and end portions of the angled struts 112 of the distal most rung, such as fifth rung 116e in the illustrated example, are forming apices 148 at the inflow end 106 of the valve 100. The angled struts 110 of the outflow rung 114 further define lower junctions 146 of the outflow rung 114, which are distal to the outflow apices 144, such that the angled struts 110 of the outflow rung 114 circumferentially extend in a zig-zagged formation between the outflow apices 144 and the lower junctions 146 of the outflow rung 114.

The upper end portion of angled struts 112 of the proximal-most or first subsequent rung 116a intersect at upper junctions 150 of the first subsequent rung 116a. In some examples, at least some of the upper junctions 150 of the first subsequent rung 116a are free apices 152, defined as junctions or apices of the first subsequent rung 116a which are not attached to any other struts 108 other than the angled struts 110 of the first subsequent rung 116a. The remainder of the upper junctions 150 of the first subsequent rung 116a, which are not free apices 152, can be further connected to axial struts 126 extending proximally therefrom. The struts 108 can intersect at additional intermediate junctions 154 formed between the outflow apices 144 and the inflow apices 148.

The valvular structure 170 can include a plurality of leaflets 172 (e.g., three leaflets), positioned at least partially within the frame 102, and configured to regulate flow of blood through the prosthetic valve 100 from the inflow end 106 to the outflow end 104. While three leaflets 172 arranged to collapse in a tricuspid arrangement, are shown in the example illustrated in FIG. 1A, it will be clear that a prosthetic valve 100 can include any other number of leaflets 172. Adjacent leaflets 172 can be arranged together to form commissures 124 that are coupled (directly or indirectly) to respective portions of the frame 102, thereby securing at least a portion of the valvular structure 170 to the frame 102. In some examples, the valvular structure 170 can be formed as a unitary component having dedicated regions thereof defining integrally formed leaflet 172 that can be continuously interconnected at commissure attachment regions 180. Such commissure attachment regions 180 can be secured to the frame 102 to form commissures 124.

The valvular structure 170 can include, in some examples, a plurality of valvular segments 171 (e.g., three valvular segments), each valvular segment comprising one of the plurality of leaflets 172. In some examples, the plurality of valvular segments can be integrally formed as regions of a one-piece valvular structure 170, meaning that all of the valvular segments 171 are continuous with each other without the need to otherwise couple them to each other (such as by suturing, adhering, and the like) to form the valvular structure 170. In some examples, the plurality of valvular segments can be formed and provided as separate components, which can be in turn joined to each other and/or to the frame 102 (e.g., by suturing) to form the valvular structure 170. In some examples, each valvular segment 171 comprises a leaflet 172 and can further include regions that can be part of commissure attachment regions, as well as regions that can be used to couple the valvular segment 171 to the frame 102. It is to be understood that while three valvular segment 171a, 171b and 171c are illustrated in FIG. 1A, a valvular structure 170 can include, in some examples, any other number of valvular segments, such as two or more than three valvular segments.

The valvular segments 171 and leaflets 172 defined thereby can be made from, in whole or part, biological material (e.g., pericardium), bio-compatible synthetic materials, or other such materials. Further details regarding transcatheter prosthetic valves, including the manner in which the valvular structure 170 can be coupled to the frame 102 of the prosthetic valve 100, can be found, for example, in U.S. Patent Nos. 6,730,118, 7,393,360, 7,510,575, 7,993,394, 8,652,202, and 11,135,056, all of which are incorporated herein by reference in their entireties.

The prosthetic valve 100 can further comprise at least one skirt or sealing member (not annotated in FIG. 1A). In some examples, a prosthetic valve can include an inner skirt (not annotated) secured to an inner surface of the frame 102, and configured to function, for example, as a sealing member to prevent or decrease perivalvular leakage. In some examples, an inner skirt can be formed as an integrally formed region of the valvular structure 170. In some examples, an inner skirt can be provided as a separate component that can further function as an anchoring region for the leaflets 172 to the frame 102, and/or function to protect the leaflets 172 against damage which may be caused by contact with the frame 102, for example during valve crimping or during working cycles of the prosthetic valve 100. In some examples, a prosthetic valve 100 can include an outer skirt (e.g., outer skirt 118 annotated in FIGS. 9-10) mounted on an outer surface of the frame 102, and configure to function, for example, as a sealing member retained between the frame 102 and the surrounding tissue of the native annulus against which the prosthetic valve is mounted, thereby reducing risk of paravalvular leakage (PVL) past the prosthetic valve 100.

Any of an inner skirt and/or outer skirt 118 can be made of various suitable biocompatible materials, such as, but not limited to, various synthetic materials (e.g., PET) or natural tissue (e.g. pericardial tissue). In some examples, an inner skirt comprises a single sheet of material that extends continuously around the inner surface of the frame 102. In some examples, the outer skirt 118 comprises a single sheet of material that extends continuously around the outer surface of the frame 102.

In some examples, frame 102 can further include a plurality of commissure posts 134 coupled to the outflow rung 114 and extending axially therefrom. In the example illustrated in FIGS. 1A-1C, the frame 102 is shown to include commissure posts 134 extending proximally from the lower junctions 146 of the outflow rung 114. Each commissure post 134 is configured to support a commissure 124 coupled thereto, such as by including an attachment feature 136 that can facilitate coupling of the commissure 124 to the frame 102. In some examples, the attachment feature 136 can include a commissure window 138 defined between two axially-extending window struts 140. In such examples, respective commissure attachment regions 180 can be radially passed through the commissure windows 138 to form commissures 124. The number of commissure posts 134 can match the number of leaflets 172. In some examples, the frame 102 can include three commissure posts 134.

It is to be understood that other forms of attachment features 136 are contemplated, such as including of one or more slits, slots, or apertures through which sutures for attachment of the commissures 124 to the posts 134 can be passed. In some examples, attachments features 136 can comprise hooks (not shown). Similarly, it is to be understood that in some examples, a commissure post 134 can be devoid of an attachment feature, instead being formed as an axially extending strut around which appropriate portions of the leaflet 172 or valvular structure 170, such as commissure attachment regions 180, can be wrapped and sutured to.

In some examples, the commissure posts 134 can extend from lower junctions 146 of the outflow rung 114 beyond the outflow apices 144 in the proximal direction, terminating proximal to the outflow apices 144, in the expanded state of the frame 102. In such cases, commissure attachment regions 180 of a valvular structure 170, when coupled to the commissure posts 134 to form commissures 124, can have proximal end portion 181 which are proximal to the outflow apices 144.

The axial struts 126 can extend between upper junctions 150 of the first subsequent rung 116a and lower junctions 146 of the outflow rung 114. In some examples, the axial struts 126 comprise a plurality of first axial struts 128 and a plurality of second axial struts 130, wherein the first axial struts 128 are attached to lower junctions 146 of the outflow rung 114 from which commissure posts 134 extend, while the second axial struts 130 are attached to lower junctions 146 of the outflow rung 114 which are not connected to commissure posts 134. Stated otherwise, the first axial struts 128 are aligned with respective commissure posts 134, and can together form continuous axial frame portions passing through corresponding lower junctions 146 of the outflow rung 114, while the second axial struts 130 terminate at corresponding lower junctions 146 of the outflow rung 114 disposed between commissure posts 134. The axial struts 126, including first axial struts 128 and second axial struts 130, can be parallel to each other and/or to a central longitudinal axis of the prosthetic valve 100. In some examples, each first axial strut 128 can further include a neck portion 168 at a proximal end of the axial struts 128 that connect strut 128 to the lower junction 146 and/or the commissure post 134.

The frame 102 of the prosthetic valve 100 illustrated in FIGS. 1A-1C comprises cells 132 arranged in several rows, each row comprising a plurality of cells 132 extending circumferentially such that each cell 132 is directly coupled to two circumferentially adjacent cells 132 on both sides thereof within the same row of cells. The term "cell", as used herein, refers to a closed cell, having an enclosed perimeter defined by at least four struts 108.

The cells rows of the frame 102 can include, in some examples, an outflow row 131 of cell 132 which is the upper or proximal-most row of cells 132, positioned closer to the outflow end 104, and a plurality of subsequent rows 133 of cells 132, namely a first subsequent row 133a of cells 132, a second subsequent row 133b of cells 132, a third subsequent row 133c of cells 132, and a fourth subsequent row 133d of cells 132, wherein the first subsequent row 133a is closer to the outflow row 131, while the distal-most subsequent row, such as the fourth subsequent row 133d in the illustrated example, is closer to the inflow end. It is to be understood that four subsequent rows 133 of cells 132 are shown by way of illustration and not limitation, and that in some examples, more or less subsequent rows 133 may be defined by other frame configurations.

In some examples, the cells 132 of the outflow row 131 are coupled to adjacent cells 132 within the outflow row 131 via axial struts 126. The cells 132 of the outflow row 131 of the exemplary valve 100 shown in FIGS. 1A-1C can be generally hexagonal, each cell 132 defined between two angled struts 110 of the outflow rung 114, four angled struts 112 of the first subsequent rung 116a, and two axial struts 126 extending between the outflow rung 114 and the first subsequent rung 116a.

Cells 132 of the subsequent rows 133 can be generally diamond-shaped cells, with the cells 132 of the first subsequent row 133a defined by two angled struts 112 of the first subsequent rung 116a and two angled struts 112 of the second subsequent rung 116b, cells 132 of the second subsequent row 133b defined by two angled struts 112 of the second subsequent rung 116b and two angled struts 112 of the third subsequent rung 116c, cells 132 of the third subsequent row 133c defined by two angled struts 112 of the third subsequent rung 116c and two angled struts 112 of the fourth subsequent rung 116d, and cells 132 of the fourth subsequent row 133d defined by two angled struts 112 of the fourth subsequent rung 116d and two angled struts 112 of the fifth subsequent rung 116e.

A prosthetic valve 100 may be expanded against a calcified aortic annulus, requiring it to overcome the relatively increased rigidity of the calcified tissue during expansion. One of the factors known to affect the radial force exerted by the frame 102 of a valve 100 on the surrounding anatomy is the number of cells 132 in corresponding cell rows, wherein a greater number of cells 132 (i.e., a higher cell density) will result in a greater radial force during expansion. Thus, frames 102 of some types of prosthetic valves 100 may be provided with a relatively large number of cells 132 to increase the radial force to overcome the resistance of the calcified annular pathologies.

Some prosthetic valves may have an overall axial length, in their expanded state, that can place the row 131 of cell 132 at the level of the coronary ostia. For example, such valves 100 can be designed to have their outflow apices 144 contacting or being placed in the vicinity of the sinuses or the Sinotubular Junction (STJ) when expanded at the site of implantation. In some instances, a patient may require implantation of a coronary stent or other procedure that requires access to a coronary artery after prosthetic valve implantation. For such instances, a physician may need to access the coronary artery through the opening defined by a cell 132 of the outflow row 131 facing the coronary ostium. In some examples, the cells 132 of the outflow row 131 of the prosthetic valve 100 can have a height (measured in the axial direction) which is greater than the height of cells 132 of the subsequent rows 133, due to the axial struts 126 interconnecting the outflow rung 114 of angled struts 110 and the first subsequent rung 116a of angled struts 112. In some examples, as further illustrated in FIGS. 1A-1C, the cells 132 of the outflow row 131 can have a width (measured in the circumferential direction, such as between two neighboring axial struts 126) which is greater than the width of cells 132 of the subsequent rows 133.

The number of cells 132 of the subsequent rows 133 can be chosen to be relatively large to provide sufficient radial force during expansion against the native annulus, which may result in a smaller width of these cells 132. Taking advantage of the fact that increased radial force during valve expansion is required primarily at the region of the native annulus, corresponding to the lower portion (i.e., distal or inflow portion) of the frame, outflow row 131 can include wider cells 132 than those of the subsequent rows 133. The width of the cells 132 in the outflow row 131 can be configured to be larger than the outer diameter of a selected coronary catheter (e.g., a 6 Fr coronary catheter).

In some examples, apices of the frame 102, such as any of the outflow apices 144, inflow apices 148, and free apices 152, comprise arcuate regions defined between upper and lower curved surfaces. Each outflow apex 144 can include an arcuate region 156 defined between an upwardly convex-shaped upper curved surface 158 and an opposing lower curved surface 160 that can form an inner depression of the outflow apex 144. Similarly, each free apex 152 can include an arcuate region 162 defined between an upwardly convex-shaped upper curved surface 164 and an opposing lower curved surface 166 that can form an inner depression of the free apex 152.

In some examples, the arcuate regions 156 of the outflow apices 144 are thinner than the remainder of the corresponding angled struts 110 diverging therefrom. In some examples, the arcuate regions 162 of the free apices 152 are thinner than the remainder of the corresponding angled struts 112 diverging therefrom. The term "width", as used herein with respect to a strut or an apex, refers to a dimension of a strut or an arcuate region of an apex measured between opposing locations on opposing surfaces of the strut or apex that extend between the radially facing inner and outer surfaces of the strut or apex, respectively.

In some instances, when the leaflets are in an open state when implanted in a patient, the leaflets can contact the free apices 152. In such cases, it is beneficial for the free apices 152 to have a arcuate regions with curved outer surfaces, as illustrated in FIGS. 1A-1C. The curved outer surface is more atraumatic and may not interfere with the leaflets of the prosthetic valve as the leaflets open and close during operation of the prosthetic valve. Thus, a long term durability of the leaflets can be increased.

While all apices of the exemplary frame 102 illustrated in FIGS. 1A-1C are shown to include arcuate regions, it is to be understood that this is shown by way of illustration and not limitation, and that in some examples, any of the outflow apices 144, inflow apices 148, or free apices 152, can have any other shape, including being in the form of "pointed" apices devoid of arcuate regions, being shaped as M-shaped double-pointed apices, and the like.

In some examples, as shown in FIGS. 1A-1C, the length of an angled strut 112 of the outflow rung 114 is greater than the length of an angled strut 112 of the first subsequent rung 116a, and/or any other subsequent rung 116 of the frame 102. In some examples, the width of an angled strut 112 of the outflow rung 114 can be larger than the width of angled struts 112 of the first subsequent rung 116a, and/or any other subsequent rung 116 of the frame 102. It is to be understood that wider angled struts 112 along the outflow rung 114 are shown and described by way of illustration and not limitation, and that in some examples, all angled struts of a frame 102, including angled struts 112 of the outflow rung 114 and angled struts 112 of subsequent rungs 116, can have similar widths.

In some examples, as illustrated in FIGS. 1A-1C, each cell 132 of the outflow row 131 can span a width of two cells 132 of the first subsequent row 133a and/or any other subsequent row 133. In some examples, the outflow row 131 includes six cells 132, while any subsequent row 133 can include twelve cells 132. The first axial struts 128 and second axial struts 130 can be alternately arranged around the circumference of the outflow row 131, such that a single first axial strut 128 can be disposed between each two successive second axial struts 130, and a single second axial strut 130 can be disposed between each two successive first axial struts 128.

While all cells 132 of the subsequent rows 133 are shown in the example illustrated in FIGS. 1A-1C to have substantially the same size and shape, it is to be understood that in some examples, cells 132 of the subsequent rows 133 can have different sizes and shapes.

Each leaflet 172 includes a leaflet belly 174 (indicated, for example, in FIG. 3D) which is the movable and unattached part of the leaflet, defined between a lower cusp line 176 and an upper free edge 178 of the leaflet. In some examples, leaflet bellies 174 described herein can have a three-dimensional and concave shape, thereby resulting in increased mobility of the leaflet when the prosthetic valve is implanted in a patient. As a result, the efficiency of the prosthetic valve including the valvular structure can be improved.

In some examples, the valvular structure 170 can comprise shaped tissue material. As mentioned above, in some examples, the valvular structure 170, including leaflet bellies 174 of leaflets 172 thereof, is a single-piece three-dimensional construct formed from a single patch 200 of tissue (shown, for example, in FIG. 3A). In some examples, leaflet bellies 174 disclosed herein are not flattenable. The term "not flattenable", as used herein, means that the leaflet belly 174 cannot be flattened. That is to say, if an attempt is made to straighten out the curve of a free edge 178 (indicated, for example, in FIGS. 3B-3D) of leaflet 172, the curve will not be able to be completely straightened such that leaflet belly 174 becomes flat. This is in contrast to leaflets that are cut from a flat patch (such as a patch 200 provided in a flat configuration as shown in FIG. 3A) and are then attached (e.g., sutured) to a frame of a prosthetic valve, wherein upon removal of such leaflets from the frame they can be laid flat on a flattened surface, with their free edges being able to completely straighten in their free state. In some examples, a leaflet belly 174 which is not flattenable defines a non-developable surface. Further details regarding leaflets or leaflet bellies thereof, which are three-dimensional or not flattenable, are described in International Application No. PCT/US2022/032303, and U.S. Provisional Application No. 63/587,399, each of which is incorporated herein by reference in its entirety.

FIGS. 2A-2C show parts of an exemplary mold assembly 210 that can be used in a method for fabricating a valvular structure 170 and/or valvular segments 171 defining leaflets 172 thereof. The mold assembly can include a first template 212 and a second template 232. The first 212 and second 232 templates can have complementary surfaces, configured to sandwich a patch 200 of material, such as tissue material, therebetween, during formation and shaping of the patch 200 to assume a three-dimensional configuration, as will be described in greater detail below. In some examples, the first 212 and second 232 templates can be provided as separate components that can be coupled to each other, such as when a patch 200 is placed therebetween, for example by virtue of appropriate fasteners 252 configured to couple both templates to each other. In some examples, the first 212 and second 232 templates can be connected to each other, such as in a pivotable manner along a hinge (not shown). FIG. 2A shows a perspective view of a mold assembly 210 with first template 212 positioned above, and spaced apart from, the second template 232. FIG. 2B shows a perspective view the first template 212 inverted relative to its orientation in FIG. 2A, so as to expose the surfaces configured to contact a patch 200 during formation of a valvular structure 170. FIG. 2C shows a front perspective view of the second template 232 of FIG. 2C.

Various exemplary implementations for mold assemblies 210, prosthetic valves 100, and/or valvular structures 170 thereof, can be referred to, throughout the specification, with superscripts, for ease of explanation of features that refer to such exemplary implementations. It is to be understood, however, that any reference to structural or functional features of any device, assembly or component, without a superscript, refers to these features being commonly shared by all specific exemplary implementations that can be also indicated by superscripts. In contrast, features emphasized with respect to an exemplary implementation of any device, assembly or component, referred to with a superscript, may be optionally shared by some but not necessarily all other exemplary implementations. For example, mold assembly 200^{a}shown in FIGS. 2A-2C is an exemplary implementation of mold assembly 210, and thus includes features described for mold assembly 210 throughout the current disclosure, except that while mold assembly 210 can be used to form either the valvular structure 170 as a unitary one-piece structure, or to form separate valvular segments 171 that can be joined to form a valvular structure 170, mold assembly 200^{a}is configured to fabricate a one-piece valvular structure 170 that includes a plurality of valvular segments 171 and leaflets 172 thereof which are integrally formed as unseparated regions of the valvular structure 170.

The first template 212 of mold assembly 200^{a} can include a planar segment 214 and a plurality of raised leaflet forming portions 216, such as the three raised leaflet forming portions 216a, 216b and 216c in the illustrated example. The second template 232 of mold assembly 200^{a} can include a planar segment 234 and a plurality of recessed leaflet forming portions 236, such as the three recessed leaflet forming portions 236a, 236b and 236c in the illustrated example. While the mold assembly 200^{a} is described herein and illustrated in FIGS. 2A-2C to include three raised leaflet forming portions 216 and three recessed leaflet forming portions 236, it is to be understood that this is shown by way of illustration and not limitation, and that a mold assembly 200^{a} can include two or more than three raised 216 and recessed 236 leaflet forming portions, matching the number of leaflets 172 in the desired resulting valvular structure 170 to be mounted in a frame 102. Thus, any reference to three raised leaflet forming portions 216 or three recessed leaflet forming portions 236 of mold assembly 200^{a} can be replaced by any other number, such as two or more than three, matching the number of desired leaflets 172 of the valvular structure 170.

The raised leaflet forming portions 216 can be laterally spaced from each other, so as to define intermediate regions 226 therebetween. The recessed leaflet forming portions 236 can be similarly spaced from each other laterally, so as to define intermediate regions 246 therebetween. Each raised leaflet forming portion 216 can include one or more surfaces shaped to instill a 3D shape of a leaflet 172, wherein each recessed leaflet forming portion 236 can have a shape that is complementary to the shape of the corresponding raised leaflet forming portions 216, also including similarly shaped one or more surfaces generally parallel to the one or more surfaces of the raised leaflet forming portion 216 when the templates 212 and 232 are coupled to each other, for example.

In some examples, each raised leaflet forming portion 216 comprises a curvilinear surface 218 that can be generally convex, a first outflow surface 220 and a second outflow surface 222 that intersect with each other and with the curvilinear surface 218, wherein the curvilinear surface 218 extends from an end of a cusp line 224 that is closer to the planar segment 214, and the first 220 and second 222 outflow surfaces are closer to an end of the first template 212 that is opposite to the planar segment 214, optionally extending from intermediate regions 226 on both sides of the corresponding raised leaflet forming portion 216 towards each other. In some examples, the first outflow surface 220 and the second outflow surface 222 can be planar surfaces, In some examples, the first outflow surface 220 and the second outflow surface 222 can be curved surfaces, optionally having different surface curvatures than the curvilinear surface 218.

In some examples, each recessed leaflet forming portion 236 comprises a curvilinear surface 238 that can be generally concave, a first outflow surface 240 and a second outflow surface 242 that intersect with each other and with the curvilinear surface 238, wherein the curvilinear surface 238 extends from an end of a cusp line 244 that is closer to the planar segment 234, and the first 240 and second 242 outflow surfaces are closer to an end of the second template 232 that is opposite to the planar segment 234, optionally extending from intermediate regions 246 on both sides of the corresponding recessed leaflet forming portion 236 towards each other. In some examples, the first outflow surface 240 and the second outflow surface 242 can be planar surfaces, In some examples, the first outflow surface 240 and the second outflow surface 242 can be curved surfaces, optionally having different surface curvatures than the curvilinear surface 238.

FIGS. 3A-3D show some stages in a method of forming a valvular structure 170. FIG. 3A shows a patch 200 of material, which can be a tissue patch having a generally rectangular patch extending between side edges 202. This patch 200 can be inserted into the mold assembly 210 between the first template 212 and the second template 232, after which both templates 212, 232 can be coupled to each other sandwiching the patch 200 therebetween, forcing it to assume the shape defined by the various surfaces of the templates 212, 232. It is to be understood that the patch 200 can be first placed over the first template 212 and the second templated 232 can be then closed over the patch 200 and the first template 212, or the patch 200 can be first placed over the second template 232 and the first template 212 can be then closed over the patch 200 and the second template 232.

Coupling of the first 212 and second 232 templates to each other can be accomplished by a variety of fastening means known in the art. For example, the second template 232 can include fasteners 252 in the form of pins, as shown in FIGS. 2A-2C, configured to extend into matching holes or apertures (not annotated separately) in the first template 212, though it is to be understood that the first template 212 can include similar pin-like fasteners 252 insertable into matching holes or apertures formed in the second template 232. Pins can be replaced, in some examples, by bolts that can be inserted into the holes or apertures and tightened with corresponding nuts (not shown). It is to be understood that other suitable means for coupling the first 212 and second 232 templates to each other are contemplated, optionally allowing to apply a desired pressure to a tissue patch 200 disposed therebetween.

When the first 212 and second 232 templates are coupled to each other, the planar segment 214, raised leaflet forming portions 216, and intermediate regions 226 of the first template 212 are aligned with the planar segment 234, recessed leaflet forming portions 236, and intermediate regions 246 of the second template 232.

In some examples, when the patch 200 comprises a tissue (e.g., pericardium), a cross-linking solution may be applied to the patch 200 in order to facilitate cross-linking of the tissue. In some examples, the cross-linking solution comprises glutaraldehyde. In some examples, the cross-linking solution may be injected into the patch 200. In some examples, the patch 200 may be soaked, immersed in, or penetrated with the cross-linking solution. In some examples, glutaraldehyde may be injected into the patch 200, and examples, the patch 200 may be soaked, immersed in, or penetrated with glutaraldehyde.

In some examples, a mold assembly 210 with the patch 200 disposed therein, may be immersed into a cross-linking solution, which may comprise glutaraldehyde. In some examples, one template of the mold assembly 210, such as the second template 232, may be removed from the mold assembly 210 when the patch 200 is partially cross-linked, and the patch 200, which can have a three-dimensional form of the desired leaflets 172, meaning that the formed leaflet bellies 174 are not flattenable, may be removed from the mold assembly 210 once it is fully cross-linked. In some examples, one template of the mold assembly 210, such as the second template 232, may be removed from the mold assembly 210 while the mold assembly 210 remains immersed in the cross-linking solution or glutaraldehyde. In some examples, a spacer or a spacing layer (not shown) may be inserted between the first 212 and second 232 templates before glutaraldehyde is applied to the patch 200. In some examples, at least one of the first 212 and/or second 232 templates can include perforations (not shown) through which cross-linking solution can flow towards portions of the patch 200 placed between the templates 212, 232.

FIG. 3B shows the patch 200 removed from the mold assembly 210. Cross-linking the tissue patch 200 can result in the tissue maintaining its shape after being removed or separated from the mold assembly 210. As shown, when formed by molding assembly 200^{a}, the patch 200 is a one-piece patch that includes all three valvular segments 171a, 171b and 171c as continuous segments thereof. FIG. 3C shows an optional subsequent step of forming a tubular valvular structure 170 by rolling the 3D-shaped patch 200. For example, the side edges 202 can be brought together in a mating or otherwise abutting relationship. Once mated, both side edges 202 can be coupled to each other, such as by sewing, adhering, or otherwise attaching the side edges 202, thus resulting in a substantially cylindrical valvular structure 170 as shown in FIG. 3D.

Each leaflet 172 of the valvular structure 170, as shown in FIG. 3D, has a free edge 178 and a cusp line 176 that can have, in some examples, a curved shape, opposite to the free edge 178. The cusp line 176 of each leaflet 172 can form a single scallop that can be, for example, parabolic in shape. Commissure attachment regions 180 of the valvular structure 170 can be defined between adjacent leaflets 172, as regions of the valvular structure 170 that extend to a certain axial length from the level of the free edges 178. Each valvular segment 171 can include, in some examples, part of a commissure attachment regions 180 (on each side of the leaflet 172), such that the combined parts of the commissure attachment regions 180 of adjacent valvular segments 171 can together define a corresponding commissure attachment regions 180. A leaf-shaped leaflet belly 174 of each leaflet 172 is defined between the cusp line 176 and the free edge 178, excluding the commissure attachment regions 180.

When the valvular structure 170 is coupled to the frame 102, a line of attachment, also referred to as a scalloped line, can extend along the cusp line 176 of all leaflets 172, together forming a scalloped shaped attachment pattern that can be stitched or otherwise coupled to the frame, directly or indirectly. The scalloped line of attachment, following at least a portion of the cusp lines 176, such as parallel to and somewhat distal to the cusp lines, optionally without extending into the commissure attachment regions 180, can have an undulating, curved scalloped shape. By forming the leaflets of the valvular structure 170 with this scalloped geometry (such as along the cusp lines 176), stresses on the leaflets 172 are reduced which, in turn, improves durability of the valve 100. The leaflet belly 174 of each leaflet 172 is the part of the leaflet 172 remaining unattached to the frame or other components of the valve after assembly, configured to open and close (or coapt) during operation of the prosthetic valve 100, such as during systole and diastole.

The shape of the leaflet bellies 174 in their free state is dictated by the shapes of the raised 216 and recessed 236 leaflet forming portions of the mold assembly 210. For example, a leaflet belly 174 of leaflet 172 formed by a mold assembly 210 can have a concave region 182 matching the shape of the curvilinear surfaces 218 and 238 of the mold assembly 210, and outflow regions 184 and 186 extending between the free edge 178 and the curvilinear region 182, matching the shapes of first outflow surfaces 220, 240 and second outflow surfaces 222, 242 of the mold assembly 210, when the outflow surfaces 220, 240 and 222, 242 are planar in shape. When the surfaces 220, 240 and 222, 242 of the mold assembly 210 are curved, matching curvatures can be formed along the regions 184 and 186 of the leaflet 172. The outflow regions 184 and 186 can be either planar or curved regions.

While leaflet bellies 174 designed to be not flattenable, as described above, optionally having a three-dimensional shape combined of three intersecting regions 182, 184 and 186 having non-identical surface curvatures, it is to be understood that any other three-dimensional shape of a leaflet belly 174 is contemplated, including cup-shaped or otherwise shaped bellies 174 formed between mating three-dimensionally shaped raised 216 and recessed 236 leaflet forming portions of the mold assembly 210, which can similarly result in the leaflet belly 174 being not flattenable.

FIG. 3D shows an exemplary valvular structure 170^{a}, which can be similar to any example of a valvular structure 170 disclosed herein, except that the valvular structure 170^{a} further comprises an engagement portion 190 extending between a distal end 192 thereof and the cusp lines 176, wherein the distal end 192 of the engagement portion 190 can be circular in the cylindrical configuration of the valvular structure 170^{a} as shown in FIG. 3D, or substantially linear in a flattened configuration of the valvular structure 170^{a} as shown in FIG. 3B for example. In some examples, the engagement portion 190 can be cylindrically disposed along an inner surface of the frame 102, and coupled thereto, such as by sutures or other couplers that extend both through the scalloped line following at least a portion of the cusp lines 176, and the distal end 192 of the engagement portion 190.

When the valvular structure 170^{a} is a one-piece material that includes three integrally formed valvular segments 171, such as valvular segments 171a, 171b and 171c shown in FIG. 3D, each valvular segment 171 defines an engagement portion segment terminating with a linear distal end segment (e.g., distal end segments 192a, 192b, 192c), such that the engagement portion segments 190a, 190b, 190c of all three valvular segments 171 together define the engagement portion 190 (or combined engagement portion) of the valvular structure 170 as a whole.

It is to be understood that a reference to a distal end of an engagement portion as a linear distal end means that the distal end 192 is not curved or otherwise angled in the axial direction, even though the distal end 192 is circumferentially disposed along the circumference of the frame 102 when the valvular structure 170 is mounted therein. Stated otherwise, the distal end 192 of the engagement portion 190 is at a uniform axial distance from the free edge(s) 178, which can define a uniform height H_{V} as shown in FIG. 3D and described in further detail below. Another definition of a linear distal end 192 can refer to the distal end 192 being at a uniform axial distance from the inflow end 106 and/or from the level of the inflow apices 148. Since the cusp lines have a curved shape in the axial direction, this also means that the axial distance between the distal end 192 of the engagement portion 190 and the cusp line 176 varies along the circumference of each valvular segment 171, increasing in size from the inflow peak of the corresponding cusp line 176 towards the commissure attachment regions 180.

The engagement portion 190 can be defined between the planar segments 214 and 234 of the first 212 and second 232 templates, respectively, during formation of the valvular structure 170. Thus, in contrast to the leaflet bellies 174 which are not flattenable, the engagement portion 190 are flattenable, meaning that when the valvular structure 170 is unattached to the frame and spread over a flat surface, the engagement portion can be flattened. Having a flattenable engagement portion 190 can assist in attachment thereof to the frame 102, wherein a flattenable engagement portion 190 can be rolled into a cylindrical or semi-cylindrical shape that can conveniently cover the inner surface of the frame 102, while the leaflet bellies 174, which are movable portions that remain unattached to the frame, can be formed as portions which are not-flattenable to improve performance of the valvular structure 170 as described above.

The commissure attachment regions 180 can be defined between the intermediate regions 226 and 246 of the first 212 and second 232 templates, respectively, during formation of the valvular structure 170. The cusp lines 176 of the valvular structure 170 can be formed between the cusp lines 224 and 244 of the first 212 and second 232 templates, respectively, during formation of the valvular structure 170.

In some examples, the distal end 192 of the engagement portion 190 can extend all the way towards, or terminate in close proximity to, the inflow end 106 of the frame 102. In some examples, the distal end 192 of the engagement portion 190 can extend all the way towards, or terminate in close proximity to, the inflow apices 148. In some examples, the distal end 192 of the engagement portion 190 is parallel to the inflow end 106. Thus, the height of the engagement portion 190, defined at each circumferential point between the distal end 192 and the corresponding cusp line 176, or the distal end 192 and a lower end of a corresponding commissure attachment region 180 between neighboring leaflets 172, is varying, while the overall height H_{V} of valvular structure 170^{a}, defined between the distal end 192 of the engagement portion 190 and the level of the free edges 178, is homogenous along the entire circumference of the valvular structure 170^{a}.

An important design parameter of a prosthetic heart valve is the diameter of the crimped profile. The diameter of the crimped profile is important because it directly influences the physician's ability to advance the prosthetic heart valve through the femoral artery or vein. More particularly, a smaller profile allows for treatment of a wider population of patients, with enhanced safety.

A valvular structure 170^{a} having an engagement portion 190 terminating at distal end 192 that can be at a uniform distance from the inflow end 106, including such a distance having a zero value when the distal end 192 of the engagement portion 190 is at the level of the inflow end 106, may result in excessive tissue material along portions of the prosthetic valve 100 closer to the inflow end 106, particularly due to the increased tissue height of the engagement portion 190 at the circumferential positions of the commissure attachment regions 180. In some instances, such as when an outer skirt is added around the outer surface of the frame 102, also terminating at or in close proximity to the inflow end 106, the added tissue material of the engagement portion 190 may increase the crimped profile.

In some examples, a patch 200 from which a valvular structure 170 is formed, can be further trimmed along an undulating or scalloped-shaped line, resulting in an attachment strip which can be significantly narrower, along the circumference of the valvular structure, than the engagement portion 190 illustrated in FIG. 3D for valvular structure 170^{a}.

FIG. 4 shows an exemplary step in the method of forming a valvular structure 170, that can be performed subsequent to crosslinking of the tissue when pressed between the first 212 and second 232 templates, which includes trimming the patch 200 by a cutting tool 254, which can be a blade (e.g., a scalpel) or any other appropriate cutting device, along an undulating edge which is substantially parallel to at least part of the cusp lines 176, optionally at a uniform distance from the cusp lines 176 at least along portions of the patch 200 extending between the commissure attachment regions 180, such that the resulting valvular structure 170^{b}, shown for example in FIG. 5, includes an attachment strip 194. The valvular structure 170^{b}is can be similar to any example of a valvular structure 170 or 170^{a} disclosed herein, except that valvular structure 170^{b} includes an attachment strip 194 that terminates at an undulating or scalloped shaped terminal edge 196, which can be parallel to the cusp lines 176 at least along portions of the valvular structure 170^{b} extending circumferentially between the commissure attachment regions 180.

The attachment strips 194 are formed between the planar segments 214, 234 of the templates 212, 232 of the mold assembly 210, meaning that, as described above with respect to the engagement portion 190, the attachment strips 194 are flattenable while the leaflet bellies are not flattenable. When a valvular segment 171 is formed as a one-piece segment, such that both the leaflet belly 174 and the attachment portions 194 are formed as continuous portions of the same piece of material, a sufficient contact area of the flattenable portion of the valvular segment 171 may be required to properly hold in place the patch 200 between both templates 212, 232 during shaping of the valvular segment 171, which may lead to a relatively wide flattenable portion similar to the engagement portion 190 described above. Advantageously, adding a step of trimming the flattenable part of the valvular segment 171 to form a corresponding flattenable attachment strip 194 can result in a narrower flattenable part used for attachment to the frame, while still allowing the patch to be properly held in position inside the mold assembly to form a leaflet belly 174 which is not flattenable, prior to the step of trimming.

As shown in FIG. 5, the attachment strip 194 can have a uniform or non-uniform width W_{S} along portions of the valvular structure 170^{b} extending circumferentially between the commissure attachment regions 180. The width W_{S} of the attachment strip 194 can be defined as the shortest distance, at each respective circumferential point, between the terminal edge 196 of the attachment strip 194 and the corresponding cusp line 176. In some examples, the width W_{S} of the attachment strip 194 along circumferential portions of the valvular structure 170^{b} extending between the commissure attachment regions 180, is in a range of about 0.5 to about 3 millimeters (mm.). In some examples, the width W_{S} of the attachment strip 194 along circumferential portions of the valvular structure 170^{b} extending between the commissure attachment regions 180, is in a range of about 1 to about 2 mm.

When the valvular structure 170^{b} is a one-piece material that includes three integrally formed valvular segments 171, such as valvular segments 171a, 171b and 171c shown in FIG. 5, each valvular segment 171 defines an engagement strip segment terminating with curved terminal edge segment (e.g., terminal edge segments 196a, 196b, 196c), such that the engagement strip segments 194a, 194b, 194c of all three valvular segments 171 together define the engagement strip 194 (or combined engagement strip) of the valvular structure 170 as a whole.

A patch 200 utilized for formation of a valvular structure 170^{b} can be still provided with a rectangular shape as shown in FIG. 3A, having a portion of the patch similar to that of the engagement portion 190 terminating at a substantially linear distal end 192 when the patch 200 is pressed between the templates 212, 232 and cross-linked as described above, wherein the excess tissue material below the cusp lines 176 can serve to provide sufficient surface area, between the planar segments 214 and 234, to maintain the patch 200 properly aligned and maintained in position between both templates 212 and 232 during shaping of the patch 200.

After cross-linking to achieve the desired three-dimensional shape of the leaflets 172, with leaflet bellies 174 thereof which are not flattenable, the patch 200 can be exposed and then trimmed as shown in FIG. 4. Trimming can be performed after complete removal of the patch 200 from the mold assembly 210, or after removal of one of the templates, such as the second template, while the patch can remain spread over the first template 212 during the trimming to form the attachment strip 194 and remove excess tissue material. The width W_{S} of the attachment strip 194 can be chosen to allow for sufficient surface area through which a suture can be passed to attached the valvular structure 170^{b}, along the attachment strip 194, to the frame 102, directly or via intermediate components. Advantageously, forming an attachment strip 194 that has an undulating or scalloped-shaped terminal edge 196 reduces the amount of tissue material used to form the valvular structure 170^{b}, thereby allowing a smaller, more even crimped profile closer to the inflow end 106 of the valve.

In some examples, marking, such as ink marking or other type of marking, can be provided over the patch 200 to mark the trim-line along which the patch 200 should be cut, following the path of the desired terminal edge 196 of the resulting attachment strip 194. In some examples, at least one of the templates 212 and/or 232 can include a marking feature (not shown) to indicate the path along which the patch 200 is to be trimmed.

As mentioned above, the frame 102 can have commissure posts 134 configured to support attachment of commissure attachment regions 180 of the valvular structure thereto. In some examples, as illustrated in FIGS. 1A-1C, the commissure posts 134 can terminate at proximal ends thereof, in the expanded configuration of the frame 102, which are proximal to the outflow apices 144. When the leaflets are closed, such as during diastole, the free edges move towards each other to seal blood flow through the prosthetic valve 100. When the valvular structure 170 has a relatively uniformly linear upper end, at least in its rectangularly spread configuration (e.g., prior to rolling into a cylindrical configuration), such that the free edges 178 of the leaflets 172 are substantially at the same height or axial level as proximal ends 181 of the commissure attachment regions 180, as illustrated for both valvular structure 170^{a} in FIG. 3D and valvular structure 170^{b} in FIG. 5, the leaflets 172 can coapt along a non-planar coaptation plane (not annotated) having a relatively elongated coaptation height C1.

In some cases, the coaptation height along which the leaflets 172 can close against each other can be reduced while preserving adequate coaptation in the closed state. FIG. 6 shows an exemplary step in the method of forming a valvular structure 170, that can be performed after crosslinking of the tissue when pressed between the first 212 and second 232 templates, which includes trimming the patch 200 by a cutting tool 254, which can be a blade (e.g., a scalpel) or any other appropriate cutting device, along the upper end of the patch 200 at the regions of the leaflets 172, to cut an upper portion of the tissue material resulting in a free edge 178 that is lower or distal to the proximal end 181 of the commissure attachment regions 180.

FIG. 7 shows an exemplary valvular structure 170^{c}resulting from the upper trimming shown in FIG. 6. The valvular structure 170^{c} is similar to any example of a valvular structure 170 disclosed herein, including any example of valvular structure 170^{a} or 170^{b}, except that the free edges 178 of the valvular structure 170^{c} are axially offset from the proximal ends 181 of the commissure attachment regions 180 by an offsetting height H_{C}, which can result in a coaptation height C2 that is smaller than the coaptation height C1. The free edge 178 defines a free edge distal-most level, which is the most distal point or axial level of the free edge 178. The offsetting height H_{C} is defined as the distance between the proximal end 181 of the commissure attachment region 180 and the free edge distal-most level 179. In the example illustrated in FIGS. 6-7, the free edge 178^{c} is shown to be relatively linear in shape, such that the free edge distal-most level 179 is the same level defined along any point of the free edge 178^{c}between the corresponding commissure attachment regions 180. In some examples, the free edge 178 can have a non-linear shape, in which case, the free edge distal-most level 179 will be the lowest point or level of the free edge 178. In some examples, the offsetting height H_{C} is greater than 2 mm. In some examples, the offsetting height H_{C} is in a range of about 2 to about 5 mm.

Placement of a patch 200 that can be substantially rectangularly shaped, between templates 212 and 232 of a mold assembly 210, and cross-linking thereof, can be performed in the same manner as described above. After cross-linking to achieve the desired three-dimensional shape of the leaflets 172, having leaflet bellies 174 which are not flattenable, the patch 200 can be exposed and then trimmed along its upper portion to form free edges 178 distally offset relative to proximal or upper ends 181 of the commissure attachment regions 180. Trimming can be performed after complete removal of the patch 200 from the mold assembly 210, or after removal of one of the templates, such as the second template, while the patch can remain spread over the first template 212 during the trimming to form the attachment strip 194 and remove excess tissue material. Advantageously, distally offsetting the free edges 178 relative to proximal ends 181 of the commissure attachment regions 180 can remove excessive tissue from the upper portions of the leaflets 172 to provide increased exposed or uncovered area of the cells 132 along the outflow row 131 through which access to the coronary ostia can be gained, or such that risk of coronary ostia sequestration can be mitigated.

In some examples, a 3D shaped cross-linked patch 200 can be trimmed along the distal portion to form an undulating attachment strip 194, and along the proximal portion to form free edges 178 which are distally offset relative to proximal ends 181 of the commissure attachment regions 180, as illustrated for example in FIGS. 6-7. In some examples, a 3D shaped cross-linked patch 200 can be trimmed along the proximal portion, without trimming the distal portions, resulting in a valvular structure 170 having free edges 178 which are distally offset relative to proximal ends 181 of the commissure attachment regions 180, in a similar manner to that illustrated in FIG. 7, while having an engagement portion 190 similar to that shown in FIG. 3D.

In some examples, a valvular structure 170 can be formed by a plurality (e.g., three) of separately formed valvular segments 171 that can be coupled (e.g., sutured) to the frame 102 and to each other, together forming the valvular structure 170 of a prosthetic valve 100. FIG. 8 shows a perspective view of a first template 212^{b} of an exemplary mold assembly 210^{b}. The first template 212^{b} can be similar to any example of the first template 212, including template 212^{a}, describe herein, except that the first template 212^{b} includes a single raised leaflet forming portion 216 instead of a plurality of raised leaflet forming portions 216 illustrated in FIGS. 2A-2C. The raised leaflet forming portion 216 of first template 212^{b} can have a curvilinear surface 218 and first 220 and second 222 outflow surfaces, or have any other three-dimensional shape, as described with respect to any example of a raised leaflet forming portion 216 described herein. The first template 212^{b} can further include a planar segment 214 distal to the cusp line 224 and intermediate regions 226 at opposite sides of the raised leaflet forming portion 216. While not shown for the sake of brevity, it is to be understood that the mold assembly 210^{b} further includes a complementary second template 232^{b} that includes a single recessed leaflet forming portion 236, shaped to mate with the first template 212^{b}.

The mold assembly 210^{b} can be utilized to form individual valvular segments 171, each defining an individual leaflet 172, in the same manner described above, mutatis mutandis, including optionally trimming any of the leaflets 172 along a free edge trim-line 258 following the shape of a desired offset free edge 178, and/or along a terminal edge trim-line 256 following the undulating shape of a terminal edge (or terminal edge segment) 196 of a resulting attachment strip segment 194.

In some examples, an individual valvular segment 171 formed by a mold assembly 210^{b} can further include laterally extending tabs (not shown) at the commissure attachment regions 180, defined between the free edge 178 and the cusp line 176 of the leaflet 172. In such cases, tabs of adjacent valvular segments 171 can be joined together during assembly, so as to form commissures 124. Such tabs can be passed through a commissure window 138 and/or wrapped around a commissure post 134 to couple the commissure 124 to the frame 102.

When a valvular structure 170 includes an attachment strip 194, a suture can be passed through the attachment strip 194 to couple it to struts 108 of the frame 102 directly, or to another component, such as a skirt, which is in turn coupled to the frame 102. During working cycles of the leaflets, pulling forces applied on the tissue at regions of suture penetration into the attachment strip 194 can cause tearing of the tissue material and impact long-term durability. In some examples, a reinforcement member 120 is added to a surface of the attachment strip 194 opposite the inner surface of the frame 102, so as to distribute the pulling forces more evenly along the reinforcement member 120 at the regions of suture penetration.

FIG. 9 shows a cross-sectional view of a portion of an exemplary prosthetic valve 100^{d}, in which an attachment strip 194 extends downwardly towards the inflow end 106 along the frame 102, and a reinforcement member 120 is placed at an inner side of the attachment strip 194, opposite to the frame 102, with a suture 122 passed through the reinforcement member 120 and the attachment strip 194 to couple them to the frame 102. In such a configuration, the terminal edge 196 is distal to the cusp line 176 when the valvular structure is mounted in the frame. In some examples, the prosthetic valve 100 can further include an outer skirt 118 disposed along an outer surface of the frame 102, in which case the suture 122 can further pass through the outer skirt 118 as well. In some examples, the reinforcement member 120 can include a strip of fabric 120^{d}, as illustrated in FIG. 9.

FIG. 10 shows a cross-sectional view of a portion of an exemplary prosthetic valve 100^{e}, in which an attachment strip 194 can be folded upwardly towards the outflow end 104 and against the inner surface of the frame 102, which can create a bending axis between the lower end portion and the remaining portion of the attachment strip 194 such that the leaflet 172 can articulate toward and away from the frame 102 during valve cycling. The bending axis of the leaflet 172 is therefore spaced inwardly from the frame 102, which can provide several advantages, including protection against leaflet abrasion during valve cycling, reduction of stress along the lower end of the valvular structure 170 during valve closure, improved blood-washing of the leaflets (thus eliminating or at least minimizing early calcification in those areas), and improved closing action of the leaflets 172. In such a configuration, the terminal edge 196 is proximal to the cusp line 176 when the valvular structure is mounted in the frame.

A reinforcement member 120 can be placed along the surface of the bent portion of the attachment strip 194 at the bending axis of the leaflet 172. In some examples, the prosthetic valve 100 can further include an outer skirt 118 disposed along an outer surface of the frame 102, in which case the suture 122 can further pass through the outer skirt 188 as well. In some examples, the reinforcement member 120 can include a thick chord or suture 120^{e}, such as a multi-filament suture (e.g., an Ethibond suture), as illustrated in FIG. 10.

It is to be understood that any type of reinforcement member 120, including in the form of a sleeve of strip of fabric 120^{d}, or in the form of a thick chord or suture 120^{e}, can be used in combination with a downwardly extending attachment strip 194 as illustrated in FIG. 9, or an upwardly folded attachment strip 194 as illustrated in FIG. 10.

FIG. 11 shows an exemplary valvular structure 170^{f}which can be trimmed along the upper end portion to define a non-zero offsetting height H_{C}, illustrated in a flattened configuration. FIG. 12 shows the valvular structure 170^{f} of FIG. 11 in its assembled or semi-cylindrical configuration. The valvular structure 170^{f} can be similar to any example of a valvular structure 170^{c} disclosed herein, except that the free edge 178^{f} of the valvular structure 170^{f} further comprises a bump or protrusion 188 that provides the leaflet 172 with excess leaflet material that can enable the leaflet to seal or close against adjacent leaflets in a prosthetic valve during operation when implanted in a patient (e.g., during diastole).

In some examples, the protrusion 188 can be centered along a central longitudinal axis of the leaflet 172, and thus can be referred to, in such cases, as a central protrusion 188. In some examples, the protrusion 188 can be offset slightly from a central longitudinal axis of the leaflet.

The bump or protrusion 188 can define a proximal peak 189, which is the proximal-most or highest point of the protrusion 188, while the portions of the free edge 178 extending between the protrusion 188 and the commissure attachment regions 180 can define the free edge distal-most level 179. In some examples, the free edge 178 can be relatively straight between the commissure attachment regions 180 and the protrusion 188. For example, the protrusion 188 can be disposed between two straight portions of the free edge 178, as illustrated in FIG. 12. In some examples, the free edge 178 can be curved between the commissure attachment regions 180 and the protrusion 188, such as by defining throughs having lowest or distal-most points that defines the free edge distal-most level 179.

A width of the protrusion 188, as well as the protrusion height H_{B} defined between the free edge distal-most level 179 and the proximal peak 189, can be sized to provide adequate closure in a desired range of valve working expansion diameters of a prosthetic valve in which it is implanted. In some examples, the width of the protrusion 188 can be equal to, or less than, about a third of the lateral length of the free edge 178 between the corresponding commissure attachment regions 180. The protrusion 188 can provide a coaptation height C3 which is greater than the coaptation height C2 of an equivalent free edge 178 which is devoid of a protrusion. For example, the coaptation height C3 of a valvular structure 170^{f}that includes a protrusion can be equal to the coaptation height C2 of an equivalent valvular structure 170^{c} devoid of a protrusion, combined with the protrusion height H_{B}.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A prosthetic valve comprising:

a frame movable between a radially compressed state and a radially expanded state; and

a valvular structure coupled to the frame and comprising a plurality of valvular segments defining leaflets configured to regulate flow through the prosthetic valve, wherein each valvular segment comprises:

a leaflet having a curved cusp line, a free edge proximal to the cusp line, and a leaflet belly extending between the cusp line and the free edge; and

an attachment strip segment extending from the cusp line to a curved terminal edge of the attachment strip segment;

wherein the attachment strip and the leaflet of each valvular segment are integrally formed from a one-piece material;

wherein the leaflet belly is not flattenable; and

wherein the attachment strip segment is flattenable.

Example 2. The prosthetic valve of any example herein, particularly of examples 1, wherein the attachment strip segment defines a width in a range of 0.5 to 3 mm.

Example 3. The prosthetic valve of any example herein, particularly of example 2, wherein the width of the attachment strip segment is in a range of 1 to 2 mm.

Example 4. The prosthetic valve of any example herein, particularly of any one of examples 2 or 3, wherein the width of the attachment strip segment is uniform.

Example 5. The prosthetic valve of any example herein, particularly of any one of examples 1 to 4, wherein the valvular structure is a one-piece material, and wherein the plurality of attachment strip segments together form a continuous one-piece attachment strip of the valvular structure.

Example 6. The prosthetic valve of any example herein, particularly of any one of examples 1 to 5, wherein the plurality of valvular segments comprises three valvular segments.

Example 7. The prosthetic valve of any example herein, particularly of any one of examples 1 to 6, wherein the valvular segments comprise tissue.

Example 8. The prosthetic valve of any example herein, particularly of example 7, wherein the tissue comprises pericardium.

Example 9. The prosthetic valve of any example herein, particularly of any one of examples 1 to 8, wherein the attachment strip segments are coupled to the frame.

Example 10. The prosthetic valve of any example herein, particularly of any one of examples 1 to 9, wherein the attachment strip segments are sutured to the frame.

Example 11. The prosthetic valve of any example herein, particularly of example 9, wherein each attachment strip segment is placed against an inner surface of the frame, such that the terminal edge is closer to an inflow end of the frame than the cusp line.

Example 12. The prosthetic valve of any example herein, particularly of example 11, further comprising a reinforcement member placed on an inner side of the attachment strip segment, opposite to the frame.

Example 13. The prosthetic valve of any example herein, particularly of example 9, wherein each attachment strip segment is folded upwardly towards an outflow end of the frame and against an inner surface of the frame, such that the terminal edge is closer to the outflow end of the frame than the cusp line.

Example 14. The prosthetic valve of any example herein, particularly of example 13, further comprising a reinforcement member placed along a surface of a bent portion of the attachment strip segment, opposite to the frame.

Example 15. The prosthetic valve of any example herein, particularly of any one of examples 12 or 14, wherein a suture connecting the attachment strip segment to the frame extends through the reinforcement member.

Example 16. The prosthetic valve of any example herein, particularly of example 15, wherein the reinforcement member comprises a strip of fabric.

Example 17. The prosthetic valve of any example herein, particularly of example 15, wherein the reinforcement member comprises a thick suture, which is thicker than the suture connecting the attachment strip segment to the frame.

Example 18. The prosthetic valve of any example herein, particularly of any one of examples 1 to 17, further comprising an outer skirt disposed around the frame, opposite to the attachment strip segments.

Example 19. The prosthetic valve of any example herein, particularly of any one of examples 1 to 18, wherein the frame comprises an outflow row of cells and a plurality of subsequent rows of cells.

Example 20. The prosthetic valve of any example herein, particularly of example 19, wherein each cell of the outflow row of cells spans a width of two cells of the subsequent rows of cells.

Example 21. The prosthetic valve of any example herein, particularly of any one of examples 19 or 20, wherein the frame further comprises an outflow rung of angled struts, and a plurality of subsequent rungs of angled struts.

Example 22. The prosthetic valve of any example herein, particularly of example 21, wherein each angled strut of the outflow rung of angled struts is longer than any angled strut of the subsequent rungs of angled struts.

Example 23. The prosthetic valve of any example herein, particularly of any one of examples 21 or 22, wherein each angled strut of the outflow rung of angled struts has a width that is greater than the width of any of the angled strut of the subsequent rungs of angled struts.

Example 24. The prosthetic valve of any example herein, particularly of any one of examples 21 to 23, wherein each cell of the outflow row of cells comprises two angled rungs of the outflow rung of angled struts, and four angled struts of a first subsequent rung of the plurality of subsequent rungs of angled struts.

Example 25. The prosthetic valve of any example herein, particularly of example 24, wherein each cell of the outflow row of cells comprises a free apex defined by the first subsequent rung of angled struts.

Example 26. The prosthetic valve of any example herein, particularly of any one of examples 21 to 25, further comprising a plurality of axial struts extending between the first subsequent rung of angled struts and the outflow rung of angled struts.

Example 27. The prosthetic valve of any example herein, particularly of example 26, wherein each cell of the outflow row of cells is defined between two of the axial struts.

Example 28. The prosthetic valve of any example herein, particularly of any one of examples 26 or 27, further comprising a plurality of commissure posts extending proximally from the outflow rung of angled struts.

Example 29. The prosthetic valve of any example herein, particularly of example 28, wherein the plurality of axial struts comprises a plurality of first axial struts and a plurality of second axial struts, wherein the first axial struts are circumferentially aligned with the commissure posts.

Example 30. The prosthetic valve of any example herein, particularly of example 29, wherein the first axial struts and the second axial struts are alternately arranged around the circumference of the frame.

Example 31. The prosthetic valve of any example herein, particularly of example 30, wherein the plurality of commissure posts comprises three commissure posts.

Example 32. The prosthetic valve of any example herein, particularly of any one of examples 30 or 31, wherein the plurality of first axial struts comprises three first axial struts, and wherein the plurality of second axial struts comprises three second axial struts.

Example 33. The prosthetic valve of any example herein, particularly of any one of examples 28 to 32, wherein each commissure post comprises an attachment feature.

Example 34. The prosthetic valve of any example herein, particularly of example 33, wherein the attachment feature comprises a commissure window defined between two window struts.

Example 35. The prosthetic valve of any example herein, particularly of any one of examples 28 to 34, wherein the valvular structure further comprises commissures formed by commissure attachment regions of the valvular structure coupled to the commissure posts of the frame.

Example 36. The prosthetic valve of any example herein, particularly of example 35, wherein the free edges are distally offset by an offsetting height defined between proximal ends of the commissure attachment regions and a distal-most level of the free edges.

Example 37. The prosthetic valve of any example herein, particularly of example 36, wherein the offsetting height is equal to or greater than 2 mm.

Example 38. The prosthetic valve of any example herein, particularly of example 37, wherein the offsetting height is not greater than 5 mm.

Example 39. The prosthetic valve of any example herein, particularly of any one of examples 1 to 38, wherein the outflow row of cells comprises six cells.

Example 40. The prosthetic valve of any example herein, particularly of example 39, wherein each of the subsequent rows of cells comprises twelve cells.

Example 41. The prosthetic valve of any example herein, particularly of any one of examples 1 to 40, wherein the plurality of subsequent rows of cells comprises four subsequent rows of cells.

Example 42. The prosthetic valve of any example herein, particularly of any one of examples 1 to 41, wherein each leaflet belly comprises a concave region extending proximally from the cusp line.

Example 43. The prosthetic valve of any example herein, particularly of example 42, wherein each leaflet belly further comprises two outflow regions extending between the concave region and the free edge.

Example 44. A method comprising:

placing a tissue patch between a first template and a second template of a mold assembly;

forming the tissue patch to include at least one leaflet belly which is not flattenable and at least one attachment strip segment with is flattenable, wherein forming the leaflet belly comprises:

pressing a first part of the tissue patch between a raised leaflet forming portion of the first template and a recessed leaflet forming portion of the second template; and

applying a cross-linking solution to the tissue patch;

and wherein the forming the attachment strip segment comprises:

trimming a second part of the tissue patch that was pressed between a planar segment of the first template and a planar segment of a second template, along a curved terminal edge, such that the resulting attachment strip segment extends between the curved terminal edge and a cusp line that separates between the attachment strip segment and the leaflet belly.

Example 45. The method of any example herein, particularly of example 44, wherein the trimming defines a width of the attachment strip segment in a range of 0.5 to 3 mm.

Example 46. The method of any example herein, particularly of example 44, wherein the trimming defines a width of the attachment strip segment in a range of 1 to 2 mm.

Example 47. The method of any example herein, particularly of any one of examples 45 or 46, wherein the width of the attachment strip segment is uniform.

Example 48. The method of any example herein, particularly of any one of examples 44 to 47, wherein the tissue patch is rectangularly shaped prior to the placing.

Example 49. The method of any example herein, particularly of any one of examples 44 to 48, wherein the placing the tissue patch comprises placing the tissue patch over the first template.

Example 50. The method of any example herein, particularly of any one of examples 44 to 49, wherein the pressing comprises attaching the first template to the second template while the tissue patch is sandwiched therebetween.

Example 51. The method of any example herein, particularly of any one of examples 44 to 50, wherein the cross-linking solution comprises glutaraldehyde.

Example 52. The method of any example herein, particularly of any one of examples 44 to 51, wherein the applying the cross-linking solution comprises injecting the cross-linking solution into the tissue patch.

Example 53. The method of any example herein, particularly of any one of examples 44 to 51, wherein the applying the cross-linking solution comprises soaking or immersing the tissue patch in the cross-linking solution.

Example 54. The method of any example herein, particularly of any one of examples 44 to 51, wherein the applying the cross-linking solution comprises allowing the cross-linking solution to flow through perforation extending through at least one of the first and second templates, towards the tissue patch.

Example 55. The method of any example herein, particularly of any one of examples 44 to 51, wherein the applying the cross-linking solution is performed while the tissue patch is retained between the first and second templates.

Example 56. The method of any example herein, particularly of any one of examples 44 to 55, wherein the first template comprises a plurality of raised leaflet forming portions and the second template comprises a plurality of recessed leaflet forming portions.

Example 57. The method of any example herein, particularly of example 56, wherein the tissue patch is a one-piece tissue patch, and wherein the at least one leaflet belly comprises a plurality of leaflet bellies of the one-piece tissue patch.

Example 58. The method of any example herein, particularly of example 57, wherein the plurality of raised leaflet forming portions comprises three raised leaflet forming portions, wherein the plurality of recessed leaflet forming portions comprises three recessed leaflet forming portions, and wherein the plurality of leaflet bellies comprises three leaflet bellies.

Example 59. The method of any example herein, particularly of any one of examples 57 or 58, wherein the at least one attachment strip segment comprises a plurality of attachment strip segments together defining a continuous attachment strip of the one-piece tissue patch.

Example 60. The method of any example herein, particularly of example 59, further comprising forming a valvular structure from the one-piece tissue patch by coupling side edges of the tissue patch to each other.

Example 61. The method of any example herein, particularly of any one of examples 44 to 60, wherein the tissue patch comprises pericardium.

Example 62. The method of any example herein, particularly of any one of examples 44 to 61, further comprising coupling the attachment strip segments to a frame of a prosthetic valve.

Example 63. The method of any example herein, particularly of example 62, wherein the coupling comprises placing each attachment strip segment, against an inner surface of the frame, such that the terminal edge is closer to an inflow end of the frame than the cusp line.

Example 64. The method of any example herein, particularly of example 62, wherein the coupling comprises folding each attachment strip segment upwardly towards an outflow end of the frame and against an inner surface of the frame, such that the terminal edge is closer to the outflow end of the frame than the cusp line.

Example 65. The method of any example herein, particularly of any one of examples 63 or 64, wherein the coupling comprises passing a suture that connect the attachment strip segment to the frame, through a reinforcement member placed against a side of the attachment strip segment opposite to the frame.

Example 66. The method of any example herein, particularly of example 65, wherein the reinforcement member comprises a strip of fabric.

Example 67. The method of any example herein, particularly of example 65, wherein the reinforcement member comprises a thick suture, which is thicker than the suture connecting the attachment strip segment to the frame.

Example 68. The method of any example herein, particularly of example 60, further comprising forming commissures by attaching commissure attachment regions of the valvular structure to commissure posts of the frame.

Example 69. The method of any example herein, particularly of any one of examples 44 to 68, wherein each raised leaflet forming portion comprises a curvilinear surface, and wherein each recessed leaflet forming portion comprises a curvilinear surface configured to mate with the curvilinear surface of the raised leaflet forming portion.

Example 70. The method of any example herein, particularly of example 69, wherein each raised leaflet forming portion further comprises a first outflow surface and a second outflow surface intersecting with each other and with the curvilinear surface of the raised leaflet forming portion, and wherein each recessed leaflet forming portion further comprises a first outflow surface and a second outflow surface, configured to mate with the corresponding first outflow surface and a second outflow surface of the raised leaflet forming portion.

Example 71. A prosthetic valve comprising:

a frame movable between a radially compressed state and a radially expanded state, the frame comprising:

an outflow rung of angled struts extending between outflow apices and lower junctions of the outflow rung; and

a plurality of commissure posts proximally extending from the lower junctions of the outflow rung; and

a valvular structure coupled to the frame and comprising a plurality of leaflets configured to regulate flow through the prosthetic valve, wherein each leaflet comprises:

a cusp line;

a free edge proximal to the cusp line; and

a leaflet belly extending between the cusp line and the free edge;

wherein the leaflet belly is not flattenable;

wherein the valvular structure further comprises a plurality of commissure attachment regions between adjacent leaflet bellies, each commissure attachment region terminating at a proximal end;

wherein the commissure attachment regions are coupled to the commissure posts of the frame to define commissures of the valvular structure; and

wherein the free edges of the leaflets are distally offset by an offsetting height defined between the proximal ends of the commissure attachment regions and a distal-most level of the free edges.

Example 72. The prosthetic valve of any example herein, particularly of example 71, wherein the offsetting height is equal to or greater than 2 mm.

Example 73. The prosthetic valve of any example herein, particularly of example 72, wherein the offsetting height is not greater than 5 mm.

Example 74. The prosthetic valve of any example herein, particularly of any one of examples 71 to 73, wherein the valvular structure is a one-piece material.

Example 75. The prosthetic valve of any example herein, particularly of any one of examples 71 to 74, wherein the plurality of leaflets comprises three leaflets.

Example 76. The prosthetic valve of any example herein, particularly of any one of examples 71 to 75, wherein the leaflets comprise tissue.

Example 77. The prosthetic valve of any example herein, particularly of example 76, wherein the tissue comprises pericardium.

Example 78. The prosthetic valve of any example herein, particularly of any one of examples 71 to 77, wherein the frame comprises an outflow row of cells and a plurality of subsequent rows of cells.

Example 79. The prosthetic valve of any example herein, particularly of example 78, wherein each cell of the outflow row of cells spans a width of two cells of the subsequent rows of cells.

Example 80. The prosthetic valve of any example herein, particularly of any one of examples 78 or 79, wherein the frame further comprises a plurality of subsequent rungs of angled struts.

Example 81. The prosthetic valve of any example herein, particularly of example 80, wherein each angled strut of the outflow rung of angled struts is longer than any angled strut of the subsequent rungs of angled struts.

Example 82. The prosthetic valve of any example herein, particularly of any one of examples 80 or 81, wherein each angled strut of the outflow rung of angled struts has a width that is greater than the width of any of the angled strut of the subsequent rungs of angled struts.

Example 83. The prosthetic valve of any example herein, particularly of any one of examples 80 to 82, wherein each cell of the outflow row of cells comprises two angled rungs of the outflow rung of angled struts, and four angled struts of a first subsequent rung of the plurality of subsequent rungs of angled struts.

Example 84. The prosthetic valve of any example herein, particularly of example 83, wherein each cell of the outflow row of cells comprises a free apex defined by the first subsequent rung of angled struts.

Example 85. The prosthetic valve of any example herein, particularly of any one of examples 80 to 84, further comprising a plurality of axial struts extending between the first subsequent rung of angled struts and the outflow rung of angled struts.

Example 86. The prosthetic valve of any example herein, particularly of example 85, wherein each cell of the outflow row of cells is defined between two of the axial struts.

Example 87. The prosthetic valve of claim 86, wherein the plurality of axial struts comprises a plurality of first axial struts and a plurality of second axial struts, wherein the first axial struts are circumferentially aligned with the commissure posts.

Example 88. The prosthetic valve of any example herein, particularly of example 87, wherein the first axial struts and the second axial struts are alternately arranged around the circumference of the frame.

Example 89. The prosthetic valve of any example herein, particularly of example 88, wherein the plurality of commissure posts comprises three commissure posts.

Example 90. The prosthetic valve of any example herein, particularly of any one of examples 88 or 89, wherein the plurality of first axial struts comprises three first axial struts, and wherein the plurality of second axial struts comprises three second axial struts.

Example 91. The prosthetic valve of any example herein, particularly of any one of examples 71 to 90, wherein each commissure post comprises an attachment feature.

Example 92. The prosthetic valve of any example herein, particularly of example 91, wherein the attachment feature comprises a commissure window defined between two window struts.

Example 93. The prosthetic valve of any example herein, particularly of any one of examples 78 to 90, wherein the outflow row of cells comprises six cells.

Example 94. The prosthetic valve of any example herein, particularly of example 93, wherein each of the subsequent rows of cells comprises twelve cells.

Example 95. The prosthetic valve of any example herein, particularly of any one of examples 78 to 90, wherein the plurality of subsequent rows of cells comprises four subsequent rows of cells.

Example 96. The prosthetic valve of any example herein, particularly of any one of examples 71 to 95, wherein each leaflet belly comprises a concave region extending proximally from the cusp line.

Example 97. The prosthetic valve of any example herein, particularly of example 96, wherein each leaflet belly further comprises two outflow regions extending between the concave region and the free edge.

Example 98. A method comprising:

placing a tissue patch between a first template and a second template of a mold assembly;

forming at least one leaflet belly between commissure attachment regions of the tissue patch by pressing part the tissue patch between a raised leaflet forming portion of the first template and a recessed leaflet forming portion of the second template, and applying a cross-linking solution to the tissue patch, such that the resulting leaflet belly is not flattenable;

trimming the leaflet patch between the commissure attachment regions on both sides of the leaflet belly, thus forming a free edge of the leaflet belly which is distally offset by an offsetting height from proximal ends of the commissure attachment regions; and

attaching the commissure attachment regions to commissure posts of a frame of a prosthetic valve, wherein the commissure posts extend proximally from lower junctions of an outflow rung of angled struts of the frame.

Example 99. The method of any example herein, particularly of example 98, wherein the offsetting height is equal to or greater than 2 mm.

Example 100. The method of any example herein, particularly of example 99, wherein the offsetting height is not greater than 5 mm.

Example 101. The method of any example herein, particularly of any one of examples 98 to 100, wherein the tissue patch is rectangularly shaped prior to the placing.

Example 102. The method of any example herein, particularly of any one of examples 98 to 101, wherein the placing the tissue patch comprises placing the tissue patch over the first template.

Example 103. The method of any example herein, particularly of any one of examples 98 to 102, wherein the pressing comprises attaching the first template to the second template while the tissue patch is sandwiched therebetween.

Example 104. The method of any example herein, particularly of any one of examples 98 to 103, wherein the cross-linking solution comprises glutaraldehyde.

Example 105. The method of any example herein, particularly of any one of examples 98 to 104, wherein the applying the cross-linking solution comprises injecting the cross-linking solution into the tissue patch.

Example 106. The method of any example herein, particularly of any one of examples 98 to 104, wherein the applying the cross-linking solution comprises soaking or immersing the tissue patch in the cross-linking solution.

Example 107. The method of any example herein, particularly of any one of examples 98 to 104, wherein the applying the cross-linking solution comprises allowing the cross-linking solution to flow through perforation extending through at least one of the first and second templates, towards the tissue patch.

Example 108. The method of any example herein, particularly of any one of examples 98 to 104, wherein the applying the cross-linking solution is performed while the tissue patch is retained between the first and second templates.

Example 109. The method of any example herein, particularly of any one of examples 98 to 108, wherein the first template comprises a plurality of raised leaflet forming portions and the second template comprises a plurality of recessed leaflet forming portions.

Example 110. The method of any example herein, particularly of example 109, wherein the tissue patch is a one-piece tissue patch, and wherein the at least one leaflet belly comprises a plurality of leaflet bellies of the one-piece tissue patch.

Example 111. The method of any example herein, particularly of example 110, wherein the plurality of raised leaflet forming portions comprises three raised leaflet forming portions, wherein the plurality of recessed leaflet forming portions comprises three recessed leaflet forming portions, and wherein the plurality of leaflet bellies comprises three leaflet bellies.

Example 112. The method of any example herein, particularly of example 111, further comprising, after the trimming and before the attaching the commissure attachment regions, forming a valvular structure from the one-piece tissue patch by coupling side edges of the tissue patch to each other.

Example 113. The method of any example herein, particularly of any one of examples 98 to 112, wherein the tissue patch comprises pericardium.

Example 114. The method of any example herein, particularly of any one of examples 98 to 113, wherein each raised leaflet forming portion comprises a curvilinear surface, and wherein each recessed leaflet forming portion comprises a curvilinear surface configured to mate with the curvilinear surface of the raised leaflet forming portion.

Example 115. The method of any example herein, particularly of example 114, wherein each raised leaflet forming portion further comprises a first outflow surface and a second outflow surface intersecting with each other and with the curvilinear surface of the raised leaflet forming portion, and wherein each recessed leaflet forming portion further comprises a first outflow surface and a second outflow surface, configured to mate with the corresponding first outflow surface and a second outflow surface of the raised leaflet forming portion.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve comprising:
a frame movable between a radially compressed state and a radially expanded state; and
a valvular structure coupled to the frame and comprising a plurality of valvular segments defining leaflets configured to regulate flow through the prosthetic valve, wherein each valvular segment comprises:
a leaflet having a curved cusp line, a free edge proximal to the cusp line, and a leaflet belly extending between the cusp line and the free edge; and
an attachment strip segment extending from the cusp line to a curved terminal edge of the attachment strip segment;
wherein the attachment strip and the leaflet of each valvular segment are integrally formed from a one-piece material;
wherein the leaflet belly is not flattenable; and
wherein the attachment strip segment is flattenable.

2. The prosthetic valve of claim 1, wherein the attachment strip segment defines a width in a range of 0.5 to 3 mm.

3. The prosthetic valve of claim 2, wherein the width of the attachment strip segment is in a range of 1 to 2 mm.

4. The prosthetic valve of claim 1, wherein the valvular structure is a one-piece material, and wherein the plurality of attachment strip segments together form a continuous one-piece attachment strip of the valvular structure.

5. The prosthetic valve of claim 1, wherein the attachment strip segments are coupled to the frame.

6. The prosthetic valve of claim 5, wherein each attachment strip segment is placed against an inner surface of the frame, such that the terminal edge is closer to an inflow end of the frame than the cusp line.

7. The prosthetic valve of claim 5, wherein each attachment strip segment is folded upwardly towards an outflow end of the frame and against an inner surface of the frame, such that the terminal edge is closer to the outflow end of the frame than the cusp line.

8. The prosthetic valve of claim 1, wherein the frame comprises a plurality of commissure posts extending proximally from an outflow rung of angled struts of the frame, and wherein the valvular structure further comprises commissures formed by commissure attachment regions of the valvular structure coupled to the commissure posts of the frame.

9. The prosthetic valve of claim 8, wherein the free edges are distally offset by an offsetting height defined between proximal ends of the commissure attachment regions and a distal-most level of the free edges.

10. The prosthetic valve of claim 9, wherein the offsetting height is equal to or greater than 2 mm.

11. The prosthetic valve of claim 10, wherein the offsetting height is not greater than 5 mm.

12. A method comprising:
placing a tissue patch between a first template and a second template of a mold assembly;
forming the tissue patch to include at least one leaflet belly which is not flattenable and at least one attachment strip segment with is flattenable, wherein forming the leaflet belly comprises:
pressing a first part of the tissue patch between a raised leaflet forming portion of the first template and a recessed leaflet forming portion of the second template; and
applying a cross-linking solution to the tissue patch;
and wherein the forming the attachment strip segment comprises:
trimming a second part of the tissue patch that was pressed between a planar segment of the first template and a planar segment of a second template, along a curved terminal edge, such that the resulting attachment strip segment extends between the curved terminal edge and a cusp line that separates between the attachment strip segment and the leaflet belly.

13. The method of claim 12, wherein the trimming defines a width of the attachment strip segment in a range of 0.5 to 3 mm.

14. The method of claim 12, wherein the first template comprises a plurality of raised leaflet forming portions and the second template comprises a plurality of recessed leaflet forming portions.

15. The method of claim 14, wherein the tissue patch is a one-piece tissue patch, and wherein the at least one leaflet belly comprises a plurality of leaflet bellies of the one-piece tissue patch.

16. The method of claim 15, wherein the at least one attachment strip segment comprises a plurality of attachment strip segments together defining a continuous attachment strip of the one-piece tissue patch.

17. The method of claim 12, further comprising coupling the attachment strip segments to a frame of a prosthetic valve.

18. The method of claim 17, wherein the coupling comprises placing each attachment strip segment, against an inner surface of the frame, such that the terminal edge is closer to an inflow end of the frame than the cusp line.

19. A prosthetic valve comprising:
a frame movable between a radially compressed state and a radially expanded state, the frame comprising:
an outflow rung of angled struts extending between outflow apices and lower junctions of the outflow rung; and
a plurality of commissure posts proximally extending from the lower junctions of the outflow rung; and
a valvular structure coupled to the frame and comprising a plurality of leaflets configured to regulate flow through the prosthetic valve, wherein each leaflet comprises :
a cusp line;
a free edge proximal to the cusp line; and
a leaflet belly extending between the cusp line and the free edge;
wherein the leaflet belly is not flattenable;
wherein the valvular structure further comprises a plurality of commissure attachment regions between adjacent leaflet bellies, each commissure attachment region terminating at a proximal end;
wherein the commissure attachment regions are coupled to the commissure posts of the frame to define commissures of the valvular structure; and
wherein the free edges of the leaflets are distally offset by an offsetting height defined between the proximal ends of the commissure attachment regions and a distal-most level of the free edges.

20. The prosthetic valve of claim 19, wherein the offsetting height is equal to or greater than 2 mm.

21. The prosthetic valve of claim 20, wherein the offsetting height is not greater than 5 mm.

22. The prosthetic valve of claim 19, wherein the valvular structure is a one-piece material.
